SEQUENCE LISTING (22 sequences)

SEQ ID NO: 1 - DNA, 20 bp, Artificial sequence
  GFAP sense primer
actcccgacccgagtggatt

SEQ ID NO: 2 - DNA, 22 bp, Artificial sequence
  GFAP antisense primer
tagacgtctgccagcttggtgg

SEQ ID NO: 3 - DNA, 25 bp, Artificial sequence
  MAP2 sense primer
gttctatctcttcttcagcacggcg

SEQ ID NO: 4 - DNA, 21 bp, Artificial sequence
  MAP2 antisense primer
cggcaccaagatggcagactt

SEQ ID NO: 5 - DNA, 20 bp, Artificial sequence
  Nestin sense primer
cagggcagcgttggaacaga

SEQ ID NO: 6 - DNA, 25 bp, Artificial sequence
  Nestin antisense primer
tctcagcctccaggagggtcctgta

SEQ ID NO: 7 - DNA, 20 bp, Artificial sequence
  Sox-2 sense primer
cgtcaagcggcccatgaatg

SEQ ID NO: 8 - DNA, 20 bp, Artificial sequence
  Sox-2 antisense primer
tcgatgaacggccgcttctc

SEQ ID NO: 9 - DNA, 24 bp, Artificial sequence
  GAPDH sense primer
ccctggccaaggtcatccatgaca

SEQ ID NO: 10 - DNA, 24 bp, Artificial sequence
  GAPDH antisense primer
aaaggagtgaggccctgcagcgta

SEQ ID NO: 11 - DNA, 21 bp, Artificial sequence
  Tyrosine hydroxylase sense primer
tcatcacctggtcaccaagtt

SEQ ID NO: 12 - DNA, 18 bp, Artificial sequence
  Tyrosine hydroxylase antisense primer
ggtcgccgtgcctgtact

SEQ ID NO: 13 - DNA, 20 bp, Artificial sequence
  Peripherin sense primer
gcgctcaagcagaggttgga

SEQ ID NO: 14 - DNA, 20 bp, Artificial sequence
  Peripherin antisense primer
ttcgcggcgatgctctcgta

SEQ ID NO: 15 - DNA, 22 bp, Artificial sequence
  CNPase sense primer
ggacaagcctgagctgcatttt

SEQ ID NO: 16 - DNA, 20 bp, Artificial sequence
  CNPase antisense primer
aattctgatgtcccggcggc

SEQ ID NO: 17 - DNA, 20 bp, Artificial sequence
  Cholineacetyltransferase sense primer
agcacttccctggcaccgat

SEQ ID NO: 18 - DNA, 22 bp, Artificial sequence
  Cholineacetyltransferase antisense primer
aggctacgatgacgtgctcagg

SEQ ID NO: 19 - DNA, 22 bp, Artificial sequence
  Notch 1 sense primer
gagtgccagctgatgccaaatg

SEQ ID NO: 20 - DNA, 21 bp, Artificial sequence
  Notch 1 antisense primer
ttgccattgacagggttggtg

SEQ ID NO: 21 - DNA, 20 bp, Artificial sequence
  Sox-1 sense primer
ccaaccaggaccgggtcaaa

SEQ ID NO: 22 - DNA, 23 bp, Artificial sequence
  Sox-1 antisense primer
ccttcttgagcagcgtcttggtc

(19) United States
(12) Patent Application Publication
(10) Pub. No.: US 2007/0264712 A1
(43) Pub. Date: Nov. 15, 2007

(54) Expansion and differentiation of neural stem cells under low oxygen conditions

(75) Inventor: Smita Savant-Bhonsale, Ellicott City, MD (US)

Correspondence Address: SEED INTELLECTUAL PROPERTY LAW GROUP PLLC, 701 FIFTH AVE, SUITE 5400, SEATTLE, WA, 98104, US

(73) Assignee: THERADIGM, INC., Sunnyvale, CA (US)

(21) Appl. No.: 11/651,648

(22) Filed: Jan. 9, 2007

Related U.S. Application Data
(60) Provisional application No. 60/757,785, filed on Jan. 10, 2006.
(60) Provisional application No. 60/817,264, filed on Jun. 28, 2006.

(51) Int. Cl. C12N 5/08; A61P 25/00; C12N 5/02

(52) U.S. Cl. 435/377; 435/368

(57) ABSTRACT

The present invention encompasses methods and compositions for enhancing the growth of neural stem cells (NSCs). The invention relates to the benefits of culturing NSCs under lowered oxygen conditions as compared to environmental oxygen conditions traditionally employed in cell culture techniques.

### CROSS-REFERENCE TO RELATED APPLICATIONS

This application is entitled to priority under 35 U.S.C. § 119(e) to U.S. Provisional Patent Applications Nos. 60/757,785, filed Jan. 10, 2006 and U.S. Provisional Patent Application No. 60/817,264, filed Jun. 28, 2006, each of which is incorporated by reference herein in its entirety.

### BACKGROUND OF THE INVENTION

During development of the central nervous system “CNS”, multipotent precursor cells, also known as neural stem cells, proliferate, giving rise to transiently dividing progenitor cells that eventually differentiate into the cell types that compose the adult brain. Stem cells (from other tissues) have classically been defined as having the ability to self-renew (i.e., form more stem cells), to proliferate, and to differentiate into multiple different phenotypic lineages. In the case of neural stem cells, this includes neurons, astrocytes and oligodendrocytes. For example, Potten and Loeffler (1990, Development 110:1001-20) characterized stem cells as undifferentiated cells capable of proliferating, self-maintenance, production of a large number of differentiated functional progeny and regenerating a tissue after injury.

Neural stem cells (NSCs) have been isolated from several mammalian species, including mice, rats, pigs and humans (WO 93/01275, WO 94/09119, WO 94/10292, WO 94/16718; Cattaneo et al., 1996, Mol. Brain. Res. 42:161-66). Human CNS neural stem cells, like their rodent homologs, when maintained in a mitogen-containing (typically epidermal growth factor (EGF) or EGF plus basic fibroblast growth factor (bFGF)) and serum-free culture medium, grow in suspension culture to form aggregates of cells known as “neurospheres”. It has been observed that human neural stem cells have doubling rates of about 30 days (Cattaneo et al., 1996, Mol Brain Res. 42:161-66). Others have shown doubling times ranging from 7-14 days in the presence of FGF and EGF (Vescovi et al., 1999 Brain Pathol. 9:569-98). Upon removal of the mitogen(s), the stem cells can differentiate into neurons, astrocytes and oligodendrocytes.

In the United States, 11,000 new cases of spinal cord injury (SCl) are reported each year. The demographic most commonly affected by SCl includes young adults, usually between the ages of 16 and 40. There are an estimated 450,000 U.S. citizens whose activities are restricted due to SCl. There is no cure for SCl and current treatments are limited to the use of steroids, such as methylprednisolone, acutely following injury and ongoing physical therapy.

Tissue repair by cell transplantation has shown great promise in recent years for a number of neurological diseases including spinal cord injury (SCl). Extensive research on an array of cell types has shown that there are several potential candidates including NSCs. NSCs can expand in vitro under several different growth conditions. Upon exposure to inductive factors, expanded NSCs are capable of differentiating into a variety of central nervous system cells both in vitro and in vivo. However, the inability to grow these cells in large quantities hinders their use in clinical trials.

To repair damaged adult neural tissues successfully, fetal tissue transplant satisfies most of the required conditions including, replacing damaged neurons, forming new synaptic connections, producing neurochemically active substances like neurotransmitters and cytokines. It has been shown that transplantation of fetal brain tissue can alleviate symptoms in patients with Parkinson and SCl, but technical and ethical difficulties to obtain enough fetal tissue limits the use of this approach.

To improve the growth rate of human fetal brain stem cells, several different methods and growth factors have been used by a number of different investigators during the last decade. It has been demonstrated that basic fibroblast growth factor (bFGF) and epidermal growth factor (EGF) are needed for expansion and maintenance of human fetal neural stem cells (hNSCs). These human NSC cultures are normally grown as free floating clusters of cells (neurospheres), but the neurospheres cannot proliferate indefinitely in the presence of bFGF and EGF alone. Addition of leukemia inhibitory factor (LIF) was shown to enhance proliferation of NSCs by decreasing doubling times to 7 days (Carpenter et al, 1999, Exp. Neurol. 158:265-278) and 4.5 days (Wright et al., 2003, J. Neurochem. 86:179-795).

There remains a need to increase the rate of proliferation of neural stem cell cultures. There also remains a need to increase the number of neurons in the differentiated cell population. There further remains a need to improve the viability of neural stem cell grafts upon implantation into a host. Thus, there is a strong need for standardization of culture conditions for maximizing the proliferation and multipotentiality of NSCs. The present invention satisfies these needs.

### BRIEF SUMMARY OF THE INVENTION

The invention includes a method of in vitro expansion and maintenance of the multipotentiality of a neural stem cell (NSC). The NSC can be derived from tissues including but not limited to brain and spinal cord.

In one aspect, the method comprises culturing a NSC in low oxygen conditions ranging from about 2.5% through about 5% oxygen, wherein the low oxygen conditions increase the cellular proliferation of the NSC when compared with an otherwise identical NSC that is cultured under ambient oxygen conditions of about 20% oxygen.

In one aspect, the NSC is cultured as an adherent cell on a coated surface. Preferably, the NSC adheres to a surface coated with polyornithine and fibronectin.

In another aspect, the NSC is derived from a human.

In yet another aspect, exogenous genetic material has been introduced into the NSC.

The invention also includes a method of increasing cellular differentiation of a NSC. In one aspect, the method comprises culturing a NSC in low oxygen conditions ranging from about 2.5% through about 5% oxygen, wherein the low oxygen conditions increase the cellular differentiation of the NSC when compared with an otherwise identical NSC that is grown in ambient oxygen conditions of about 20% oxygen. In one aspect, differentiation under low oxygen conditions preferentially drives oligodendrocyte differentiation of spinal cord NSCs.

In one aspect of the invention, the cells are differentiated in low oxygen conditions in the presence of BDNF. In another aspect, the cells are differentiated in low oxygen conditions in the presence of insulin-like growth factor 1 (IGF-1).

In another aspect, the cells are differentiated in low oxygen conditions following a period a time of having been expanded in low oxygen conditions.

Also included in the invention is an isolated NSC prepared by a method of culturing a NSC in low oxygen conditions ranging from about 2.5% through about 5% oxygen, wherein the low oxygen conditions increase the cellular proliferation of said NSC when compared with an otherwise identical NSC that is cultured under ambient oxygen conditions of about 20% oxygen.

The invention also includes a differentiated isolated NSC prepared by a method of culturing a NSC in low oxygen conditions ranging from about 2.5% through about 5% oxygen, wherein the low oxygen conditions increase the cellular proliferation of the NSC when compared with an otherwise identical NSC that is cultured under ambient oxygen conditions of about 20% oxygen.

The invention includes a method of treating a mammal having a disease, disorder or condition of the central nervous system. In one aspect, the method comprises obtaining an isolated NSC from a donor, culturing the NSC in low oxygen conditions ranging from about 2.5% through about 5% oxygen, and administering the cultured NSC to the central nervous system of the mammal. Preferably, the mammal is a human.

In one aspect, the isolated NSC is allogeneic with respect to said mammal. In another aspect, the isolated NSC is autologous with respect to said mammal.

In yet another aspect, the disease, disorder or condition of the central nervous system is selected from the group consisting of a genetic disease, brain trauma, Huntington's disease, Alzheimer's disease, Parkinson's disease, spinal cord injury, stroke, multiple sclerosis, cancer, CNS lysosomal storage diseases and head trauma, epilepsy. In a further aspect, the disease, disorder or condition is injury to the tissue or cells of the central nervous system.

In another aspect, the cultured NSC administered to the central nervous system remains present and/or replicates in the central nervous system.

In one aspect, the NSC is further cultured in vitro in a differentiation medium prior to administering the NSC to the mammal in need thereof. Preferably, the NSC is differentiated in low oxygen conditions. In yet another aspect, the NSC is genetically modified prior to administering the NSC to the mammal in need thereof.

The invention also includes a composition comprising an isolated NSC and a biologically compatible lattice, wherein the NSC is prepared by a method comprising culturing said NSC in low oxygen conditions ranging from about 2.5% through about 5% oxygen on a biologically compatible lattice.

In another aspect, the composition comprises a NSC differentiated in low oxygen conditions and a biologically compatible lattice. Preferably, the NSC is derived from spinal cord. More preferably, the spinal cord NSC is differentiated in low oxygen conditions towards the oligodendrocyte lineage.

In one aspect, the lattice comprises polymeric material. In another aspect, the polymeric material comprises monomers selected from the group of monomers consisting of glycolic acid, lactic acid, propyl fumarate, caprolactone, hyaluronan, hyaluronic acid and combinations thereof. In yet another aspect, the polymeric material comprises proteins, polysaccharides, polyhydroxy acids, polyorthoesters, polyanhydrides, polyphosphazenes, synthetic polymers or combinations thereof.

In yet another aspect, the polymeric material is a hydrogel formed by crosslinking of a polymer suspension having the cells dispersed therein.

In a further aspect, the biologically compatible lattice is further coated with polyornithine. In another aspect, the biologically compatible lattice is further coated with fibronectin. In yet another aspect, the biologically compatible lattice is further coated with polyornithine and fibronectin.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1 is a schematic of a flow chart depicting the type of procedures for processing and culturing of neural stem cells (NSCs).

FIG. 2 is a graph depicting the effects of culturing NSCs cultured in four different growth conditions: uncoated flasks under ambient (about 20% oxygen) or lower oxygen (about 3% oxygen) and coated flasks under ambient or lower oxygen.

FIG. 3, comprising FIGS. 3A through 3D, is a series of images depicting the morphology of NSCs cultured under four different growth conditions. The conditions are as follows: uncoated flasks under ambient (FIG. 3A) or lower oxygen (FIG. 3C); and coated flasks under ambient (FIG. 3B) or lower oxygen (FIG. 3D).

FIG. 4, comprising FIGS. 4A through 4G is a series of FACS analysis graphs depicting the phenotype of human NSCs cultured under ambient and low oxygen conditions. FIGS. 4A through 4G depict the profile of A2B5, CD56, CD133, CD184, MHC Class I molecule, MHC Class II molecule and Nestin, respectively.

FIG. 5, comprising FIGS. 5A through 5D, is a series of images depicting the differentiation of human NSCs cultured under four different growth conditions. The conditions are as follows: uncoated flasks under ambient (FIG. 5A) or lower oxygen (FIG. 5C); and coated flasks under ambient (FIG. 5B) or lower oxygen (FIG. 5D). NSCs grown under lower oxygen differentiated into about 75-80% MAP2 positive neurons and about 18-20% astrocytes while under ambient oxygen about 65% cells differentiated into MAP2 positive neurons and about 33% into astrocytes.

FIG. 6, comprising FIGS. 6A through 6D, is a series of images depicting the morphology of NSCs cultured under different growth conditions. FIGS. 6A and 6B depict brain derived NSCs cultured under 20% O₂ and 3% O₂, respectively.

FIGS. 6C and 6D depict spinal cord derived NSCs cultured under 20% O₂ and 3% O₂, respectively.

FIG. 7, comprising FIGS. 7A through 7L is a series of FACS analysis graphs depicting the phenotype of spinal derived NSCs cultured under ambient and low oxygen conditions. FIGS. 7A through 7L depict the profile of CD90, CD9, CD29, CD146, MHC Class I molecule, A2B5, CD133, CD56, CD63, CD105, MHC Class II molecule and Nestin, respectively.

FIG. 8, comprising FIGS. 8A through 8D, is a series of images depicting the differentiation of spinal cord derived NSCs cultured under 20% O₂ (FIGS. 8A and 8B) and 3% O₂ (FIGS. 8C and 8D).

### DETAILED DESCRIPTION

In prior art methods, NSCs are typically cultured in the presence of growth factors such as basic fibroblast growth factor (bFGF) and epidermal growth factor (EGF) as free floating clusters of cells (neurospheres) in ambient oxygen levels (about 20% oxygen). According to the methods of the present invention, NSCs are cultured under low oxygen conditions. Preferably, the NSCs are cultured under low oxygen conditions as an adherent cell population on a coated surface.

The present invention comprises methods and compositions for inducing or enhancing proliferation of neural stem cells (NSCs) while preserving their multipotentiality when culturing the cells in low oxygen conditions. In another aspect, the invention includes enhancing the differentiation of NSCs into cells of the CNS including, but not limited to neurons, astrocytes, and oligodendrocytes when culturing the cells in low oxygen levels.

The present invention also relates to the discovery that the expression of Nestin molecules by NSCs is regulated by culturing the cells in low oxygen conditions. That is, the expression of Nestin molecules by NSCs is increased when cultured in low oxygen conditions.

The cells produced by the methods of invention can provide a source of partially or fully differentiated, functional cells for research, transplantation, and development of tissue engineering products for the treatment of animal disease, preferably human disease, and tissue repair or improvement.

Definitions

As used herein, each of the following terms has the meaning associated with it in this section.

The articles “a” and “an” are used herein to refer to one or to more than one (i.e. to at least one) of the grammatical object of the article. By way of example, “an element” means one element or more than one element.

The term “about” will be understood by persons of ordinary skill in the art and will vary to some extent on the context in which it is used.

“Allogeneic” refers to a graft derived from a different animal of the same species. As used herein, the term “autologous” is meant to refer to any material derived from the same individual to which it is re-introduced.

As used herein, the term “ambient oxygen levels” or otherwise “ambient oxygen conditions” refers to traditional oxygen levels used in culturing neural stem cells in traditional high incubator oxygen levels. An ambient oxygen level includes a culture condition of about 20% oxygen.

“Low oxygen levels” or otherwise “low oxygen conditions,” as used herein, refers to conditions where oxygen levels are lower than ambient oxygen levels. A low oxygen condition includes a condition where the oxygen level is less than about 20% oxygen. Preferably, the oxygen level is less than about 15% oxygen, more preferably less than about 10%, yet more preferably less than about 5%. Most preferably, the oxygen level is about 3%.

As used herein, the term “biocompatible lattice,” is meant to refer to a substrate that can facilitate formation into three-dimensional structures conducive for tissue development. Thus, for example, cells can be cultured or seeded onto such a biocompatible lattice, such as one that includes extracellular matrix material, synthetic polymers, cytokines, growth factors, etc. The lattice can be molded into desired shapes for facilitating the development of tissue types. Also, at least at an early stage during culturing of the cells, the medium and/or substrate is supplemented with factors (i.e., growth factors, cytokines, extracellular matrix material, etc.) that facilitate the development of appropriate tissue types and structures.

As used herein, “central nervous system” should be construed to include brain and/or the spinal cord of a mammal. The term may also include the eye and optic nerve in some instances.

The term “coated” is used herein to refer to a surface that has been treated with an extracellular component. The coated surface provides a surface on which cells may adhere. Examples of an extracellular component include but not limited to fibronectin, laminin, poly-D-lysine and poly-L-lysine.

As used herein, the term “disease, disorder or condition of the central nervous system” is meant to refer to a disease, disorder or a condition which is caused by a genetic mutation in a gene that is expressed by cells of the central nervous system such that one of the effects of such a mutation is manifested by abnormal structure and/or function of the central nervous system, such as, for example, neurodegenerative disease or primary tumor formation. Such genetic defects may be the result of a mutated, non-functional or under-expressed gene in a cell of the central nervous system. The term should also be construed to encompass other pathologies in the central nervous system which are not the result of a genetic defect per se in cells of the central nervous system, but rather are the result of infiltration of the central nervous system by cells which do not originate in the central nervous system, for example, metastatic tumor formation in the central nervous system. The term should also be construed to include trauma to the central nervous system induced by direct injury to the tissues of the central nervous system.

“Differentiated” is used herein to refer to a cell that has achieved a terminal state of maturation such that the cell has developed fully and demonstrates biological specialization and/or adaptation to a specific environment and/or function. Typically, a differentiated cell is characterized by expression of genes that encode differentiation associated proteins in that cell. When a cell is said to be “differentiating,” as that term is used herein, the cell is in the process of being differentiated.

“Differentiation medium” is used herein to refer to a cell growth medium comprising an additive or a lack of an additive such that a stem cell, embryonic stem cell, ES-like cell, neurosphere, NSC or other such progenitor cell, that is not fully differentiated, when incubated in the medium, develops into a cell with some or all of the characteristics of a differentiated cell.

“Expandability” is used herein to refer to the capacity of a cell to proliferate for example to expand in number, or in the case of a cell population, to undergo population doublings.

“Graft” refers to a cell, tissue, organ or otherwise any biological compatible lattice for transplantation.

As used herein, the term “growth medium” is meant to refer to a culture medium that promotes growth of cells.

As used herein, the term “modulate” is meant to refer to any change in biological state, i.e. increasing, decreasing, and the like.

As used herein, the term “multipotential” or “multipotentiality” is meant to refer to the capability of a stem cell of the central nervous system to differentiate into more than one type of cell. For example a multipotential stem cell of the central nervous system is capable of differentiating into cells including but not limited to neurons, astrocytes and oligodendrocytes.

“Neurosphere” is used herein to refer to a neural stem cell/progenitor cell wherein nestin expression can be detected, including, inter alia, by immunostaining to detect nestin protein in the cell. Neurospheres are aggregates of proliferating neural stem/progenitor cells, and the formation of neurosphere is a characteristic feature of neural stem cells in in vitro culture.

“Neural stem cell” is used herein to refer to undifferentiated, multipotent, self-renewing neural cell. A neural stem cell is a multipotent stem cell which is able to divide and, under appropriate conditions, has self-renewal capability and can terminally differentiate into neurons, astrocytes, and oligodendrocytes. Hence, the neural stem cell is “multipotent” because stem cell progeny have multiple differentiation pathways. A neural stem cell is capable of self maintenance, meaning that with each cell division, one daughter cell will also be, on average, a stem cell. Neural stem cells can be derived from tissues including, but not limited to brain and spinal cord.

The term “derived from” is used herein to mean to originate from a specified source.

“Neural cell” is used herein to refer to a cell that exhibits a morphology, a function, and a phenotypic characteristic similar to that of glial cells and neurons derived from the central nervous system and/or the peripheral nervous system.

“Neuron-like cell” is used herein to refer to a cell that exhibits a morphology similar to that of a neuron and detectably expresses a neuron-specific marker, such as, but not limited to, MAP2, neurofilament 200 kDa, neurofilament-L, neurofilament-M, synaptophysin, β-tubulin III (TUJ1), Tau, NeuN, a neurofilament protein, and a synaptic protein.

“Astrocyte-like cell” is used herein to refer to a cell that exhibits a phenotype similar to that of an astrocyte and which expresses the astrocyte-specific marker, such as, but not limited to, GFAP.

“Oligodendrocyte-like cell” is used herein to refer to a cell that exhibits a phenotype similar to that of an oligodendrocyte and which expresses the oligodendrocyte-specific marker, such as, but not limited to, O-4.

“Proliferation” is used herein to refer to the reproduction or multiplication of similar forms, especially of cells. That is, proliferation encompasses production of a greater number of cells, and can be measured by, among other things, simply counting the numbers of cells, measuring incorporation of ³H-thymidine into the cells, and the like.

“Transplant” refers to a biocompatible lattice or a donor tissue, organ or cell, to be transplanted.

As used herein, a “therapeutically effective amount” is the amount of cells which is sufficient to provide a beneficial effect to the subject to which the cells are administered.

“Xenogeneic” refers to a graft derived from an animal of a different species.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The phrase “under transcriptional control” or “operatively linked” as used herein means that the promoter is in the correct location and orientation in relation to the polynucleotides to control RNA polymerase initiation and expression of the polynucleotides.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a cell substantially only if the cell is a cell of the tissue type corresponding to the promoter.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (i.e., naked or contained in liposomes) and viruses that incorporate the recombinant polynucleotide.

### DESCRIPTION

The present invention includes a method of enhancing the proliferation of NSCs while maintaining their multipotential capacity (their capacity to differentiate into one of various cell types, such as neurons, astrocytes, oligodendrocytes and the like). Preferably, the NSCs are derived from a mammal, more preferably the NSCs are derived from a human.

The method comprises isolating NSCs using methods well known in the art and culturing NSCs under low oxygen conditions. Preferably, the NSCs are cultured under low oxygen conditions on a coated surface maintained as an adherent culture that expands into adherent and/or non-adherent neurospheres cultures. More preferably, the isolated NSCs are cultured as an adherent culture and expand into an adherent culture in low oxygen conditions.

The present invention also relates to the discovery that the expression Nestin by NSCs can be modulated by culturing NSCs according to the methods disclosed herein. The disclosure presented herein demonstrates that in addition to enhancing the proliferation of NSCs while preserving their multipotential capacities, culturing NSCs under low oxygen conditions increases expression of Nestin molecules when compared with the expression of Nestin molecules by NSCs cultured using standard methods known in the art. As such, the present invention provides a method of culturing NSCs in a manner that provides additional benefits over the standard methods used for enhancing proliferation of NSCs in culture.

The NSC culturing methods described herein solve an essential problem for the generation of NSCs for use as a treatment of human diseases. That is, prior to the disclosure provided herein, NSCs were difficult to isolate and expand in culture (i.e., it was difficult to induce them to proliferate in sufficient number for therapeutic purposes). As such, expansion of these cells using traditional methods yields a cell that is inadequate for in vitro model assay studies let alone for therapeutic purposes.

The invention includes methods and compositions for enhancing the differentiation of NSCs under low oxygen conditions. Partially or terminally differentiated cells may be characterized by the identification of surface and intracellular proteins, genes, and/or other markers indicative of the differentiated cell type. These methods include, but are not limited to, detection of cell surface proteins by immunofluorescent assays such as flow cytometry or in situ immunostaining; detection of intracellular proteins by immunofluorescent methods such as flow cytometry or in situ immunostaining; detection of the expression of lineage selective mRNAs by methods such as polymerase chain reaction, in situ hybridization, and/or other blot analysis.

Low Oxygen Conditions

The invention relates to the discovery that culturing NSCs under low oxygen conditions provides additional benefits over prior methods of culturing NSCs. Low oxygen conditions include any culturing conditions wherein the level of oxygen is below atmospheric oxygen (e.g. about 20% oxygen). In one aspect, low oxygen conditions are defined as the percent of oxygen within the range that includes about 2.5% oxygen through about 19% oxygen.

In some aspects, low oxygen conditions comprise a range that includes about 2.5% through about 19% oxygen. In other aspects, the low oxygen conditions comprise a range that includes about 2.5% through about 15% oxygen. In still other aspects, the low oxygen conditions comprise a range that includes about 2.5% through about 10% oxygen. In further aspects, the low oxygen conditions comprise a range that includes about 2.5% through about 6% oxygen. These are exemplary ranges of low oxygen conditions to be used in culture and it should be understood that those of skill in the art will be able to employ oxygen levels falling in any of these ranges generally or an oxygen level between any of these ranges that mimics physiological oxygen conditions for cells of the central nervous system. Thus, one of skill in the art could set the oxygen culture levels at 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5 or any other oxygen level between any of these figures. Preferably, the cells are cultured in 3% oxygen.

Ideally, the level of oxygen is kept as close as possible to the normal physiological oxygen conditions in which a particular cell would be found in vivo. For example, cells derived from certain regions of the brain may normally exist in oxygen conditions as low as about 1.5% oxygen. It should be noted that low oxygen conditions are not to be considered to be the same as hypoxic conditions. The low oxygen conditions are intended to mimic physiological conditions, where as “hypoxic conditions” are generally conditions where the oxygen level is less than 0.1% O₂ (Husemann et al., 1999, Neurosci Lett. 275:53-6). The low oxygen culture conditions for culturing NSCs disclosed herein provide a method to promote increased expansion of the cells, inhibit apoptosis of the cells in culture, promote differentiation of neural stem cells, and otherwise render such cells more amenable for use in transplantation.

The present invention includes a method of culturing NSCs under low oxygen conditions to enrich a population of NSCs that are expanded and/or differentiated to express a particular neuronal phenotype. The NSCs of the present invention may be proliferated under these conditions as an adherent culture. The NSCs may be subject to further enrichment using methods such as cell sorting and the like.

In one embodiment, culturing the cells in low oxygen conditions reduces the level of apoptotic and non-apoptotic cell death and therefore increases the survival of the cells. In some aspects, the increased survival of the cells may be due to both an inhibition of apoptosis and non-apoptotic death. Apoptosis or programmed cell death is a well known phenomenon and can be measured using techniques well know to those of skill in the art.

After culturing the NSCs in low oxygen conditions, the NSCs can also be differentiated in low oxygen conditions, wherein the low oxygen conditions promote increased differentiation of the cells. In some embodiments, the cells may be continuously maintained in low oxygen conditions for differentiation.

In some embodiments, the cells cultured under a low oxygen condition are transferred to a different low oxygen condition. Regardless of the oxygen conditions used for differentiating the cells, a skilled artisan can assess the level of differentiation by assessing the level of differentiation markers expressed by the cells. When the cells of the present invention are differentiated, many of them lost their nestin positive immunoreactivity. Antibodies specific for various neuronal or glial proteins may be employed to identify the phenotypic properties of the differentiated cells. Neurons may be identified using antibodies to neuron specific neurofilament, Tau, beta-tubulin, or other known neuronal markers. Astrocytes may be identified using antibodies to glial fibrillary acidic protein “GFAP”, or other known astrocytic markers. Oligodendrocytes may be identified using antibodies to galactocerebroside, O4, myelin basic protein “MBP” or other known oligodendrocytic markers. Glial cells in general may be identified by staining with antibodies, such as the M2 antibody, or other known glial markers.

In one embodiment, the method of differentiating the cells under low oxygen conditions drives preferential oligodendrocyte differentiation. Preferably, the NSCs are spinal cord NSCs. Differentiating NSCs in low oxygen conditions increases the cellular differentiation at least 2 fold when compared with an otherwise identical NSC that is differentiated under ambient oxygen conditions of about 20% oxygen.

Isolation of NSCs

NSCs can be obtained from the central nervous system of a mammal, preferably a human. These cells can be obtained from a variety of tissues including but not limited to, fore brain, hind brain, whole brain and spinal cord. NSCs can be isolated and cultured using the methods detailed elsewhere herein or using methods known in the art, for example using methods disclosed in U.S. Pat. No. 5,958,767 hereby incorporated by reference herein in its entirety. Other methods for the isolation of NSCs are well known in the art, and can readily be employed by the skilled artisan, including methods to be developed in the future. For example, NSCs have been isolated from several mammalian species, including mice, rats, pigs and humans. See, i.e., WO 93/01275, WO 94/09119, WO 94/10292, WO 94/16718 and Cattaneo et al. (1996 Mol. Brain. Res. 42:161-66), all of which are incorporated by reference herein in their entirety. The present invention is in no way limited to these or any other methods of obtaining a cell of interest.

Any suitable tissue source may be used to derive the NSCs of this invention. NSCs can be induced to proliferate and differentiate either by culturing the cells in suspension or on an adherent substrate (See, i.e., U.S. Pat. No. 5,750,376 and U.S. Pat. No. 5,753,506; both incorporated herein by reference in their entirety). In addition, the cells can be cultured in any of the medium described therein.

NSCs can be isolated from many different types of tissues, for example, from donor tissue by dissociation of individual cells from the connecting extracellular matrix of the tissue, or from commercial sources of NSCs. In one example, tissue from brain is removed using sterile procedures, and the cells are dissociated using any method known in the art including treatment with enzymes such as trypsin, collagenase and the like, or by using physical methods of dissociation such as mincing or treatment with a blunt instrument. Dissociation of neural cells, and other multipotent stem cells, can be carried out in a sterile tissue culture medium. Dissociated cells are centrifuged at low speed, between 200 and 2000 rpm, usually between 400 and 800 rpm, the suspension medium is aspirated, and the cells are then resuspended in culture medium.

Following isolation, NSCs are incubated in a culturing medium in a culture apparatus for a period of time or until the cells reach confluency before passing the cells to another culture apparatus. The culturing apparatus can be of any culture apparatus commonly used for culturing cells in vitro. Preferably, the level of confluency of the cells is greater than 70% before passing the cells to another culture apparatus. More preferably, the level of confluency of the cells is greater than 90%. A period of time can be any time suitable for the culture of cells in vitro. The culturing medium may be replaced during the culture of the NSCs at anytime. Preferably, the culture medium is replaced every 3 to 4 days. NSCs are then harvested from the culture apparatus whereupon the NSCs can be used immediately or they can be cryopreserved and stored for use at a later time. NSCs may be harvested by trypsinization, or any other procedure used to harvest cells from a culture apparatus.

Standard culture media typically contains a variety of essential components required for cell viability, including inorganic salts, carbohydrates, hormones, essential amino acids, vitamins, and the like. Preferably, DMEM or F-12 is the standard culture medium, most preferably a 50/50 mixture of DMEM and F-12. Both media are commercially available (DMEM; GIBCO, Grand Island, N.Y.; F-12, GIBCO, Grand Island, N.Y.). A premixed formulation of DMEM/F-12 is also available commercially. It is advantageous to provide additional glutamine to the medium. It is also advantageous to provide heparin in the medium. It is further advantageous to add sodium bicarbonate to the medium. It is also advantageous to add N2 supplement (Life Technologies, Gaithersburg, Md.). Preferably, the conditions for culturing the NSCs should be as close to physiological conditions as possible. The pH of the culture medium is typically between 6-8, preferably about 7, most preferably about 7.4. Cells are typically cultured at a temperature between 30-40° C., preferably between 32-38° C., most preferably between 35-37° C.

Various terms are used herein to describe cells in culture. Cell culture refers generally to cells taken from a living organism and grown under controlled condition. A primary cell culture is a culture of cells, tissues or organs taken directly from an organism and before the first subculture. Cells are expanded in culture when they are placed in a growth medium under conditions that facilitate cell growth and/or division, resulting in a larger population of the cells. When cells are expanded in culture, the rate of cell proliferation is typically measured by the amount of time required for the cells to double in number, otherwise known as the doubling time.

Each round of subculturing is referred to as a passage. When cells are subcultured, they are referred to as having been passaged. A specific population of cells, or a cell line, is sometimes referred to or characterized by the number of times it has been passaged. For example, a cultured cell population that has been passaged ten times may be referred to as a P10 culture. The primary culture, i.e., the first culture following the isolation of cells from tissue, is designated P0. Following the first subculture, the cells are described as a secondary culture (P1 or passage 1). After the second subculture, the cells become a tertiary culture (P2 or passage 2), and so on. It will be understood by those of skill in the art that there may be many population doublings during the period of passaging; therefore the number of population doublings of a culture is greater than the passage number. The expansion of cells (i.e., the number of population doublings) during the period between passaging depends on many factors, including but is not limited to the seeding density, substrate, medium, and time between passaging.

The cells employed in the methods disclosed herein may be any cells that are routinely used for CNS studies involving the CNS diseases. As such, the cells may be primary tissue culture cells or derived from a cell line. The cells may be fetal cells or adult cells. It is contemplated that the cells may be selected from the group consisting of central nervous system stem cells, spinal cord-derived progenitor cells, glial cells, astrocytes, neuronal stem cells, central nervous system neural crest-derived cells, neuronal precursor cells, neuronal cells, hepatocytes, adipose tissue derived stromal cells and bone marrow derived cells. In preferred embodiments, it is contemplated that the cells may be mecencephalic progenitor cells, lateral ganglion precursor cells, cortical precursor cells, astrocytes or neuroblasts.

Culturing of NSCs

The invention comprises methods and compositions for culturing NSCs under low oxygen conditions to enhance their proliferation rate without losing their capacity to differentiate. In one embodiment of the present invention, the cells are cultured on a surface coated with polyomithine and fibronectin. Preferably, the cells are cultured on a coated surface as an adherent cell population. However, the present invention should not be construed to include culturing the cells solely on a surface coated with polyornithine and fibronectin. Rather, the present invention should encompass any biocompatible material that can be used to culture NSCs as an adherent culture.

Without wishing to be bound by any particular theory, one benefit of culturing the cells as an adherent cell population is to obtain a more homogenous cell population than that possible when the cells are grown as a free floating cluster of cells known as neurospheres. In addition, an adherent population of cells provides a means for the cell population to be exposed more uniformly to factors (i.e. growth factors, trophic factors and the like) present in the culture medium.

In another embodiment of the present invention, as disclosed more fully elsewhere herein, the cells cultured as an adherent cell population on a coated surface under low oxygen conditions were observed to have a heightened proliferation rate without losing their capacity to differentiate into cell types including, but not limited to neurons, astrocytes, and oligodendrocytes. Preferably, the proliferation rate of the cells when cultured according to the methods of the present invention is enhanced at least about 2 fold, more preferably at least about 5 folds, even more preferably at least about 10 fold, most preferably at least about 20 fold and any full or fraction of an integer there between, where the cells do not lose their capacity to differentiate.

The invention also comprises culturing NSCs in a defined medium in a 2-dimensional or 3-dimensional biocompatible lattice. The use of a biocompatible lattice facilitates in vivo tissue engineering by supporting and/or directing the fate of the implanted cells. For example, the invention can facilitate the regeneration of brain tissue by culturing the NSCs under conditions suitable for them to expand and divide to form a desired structure. In some applications, this is accomplished by transferring them to an animal typically at a site at which the new matter is desired.

In another embodiment, the cells can be induced to differentiate and expand into a desired tissue in vitro prior to administrating the cells to a recipient. In such an application, the cells are cultured on substrates that facilitate formation into three-dimensional structures conducive for tissue development. Thus, for example, the cells can be cultured or seeded onto a bio-compatible lattice, such as one that includes extracellular matrix material, synthetic polymers, cytokines, growth factors, and the like. Such a lattice can be molded into desired shapes for facilitating the development of tissue types. Also, at least at an early stage during such culturing, the medium and/or substrate is supplemented with factors (e.g., growth factors, cytokines, extracellular matrix material, and the like) that facilitate the development of appropriate tissue types and structures. In some embodiments, it is desirable to co-culture the cells with mature cells of the respective tissue type, or precursors thereof, or to expose the cells to the respective medium, to direct differentiation to the desired cell type.

To facilitate the use of the NSCs of the present invention for producing a desired tissue, the invention provides a composition including the inventive cells (and populations) and a biologically compatible lattice. Typically, the lattice is formed from polymeric material, having fibers as a mesh or sponge, typically with spaces on the order of between about 100 μm and about 300 μm. Such a structure provides sufficient area on which the cells can grow and proliferate. Preferably, the lattice is biodegradable over time, so that it will be absorbed into the animal matter as it develops. Suitable polymeric lattices, thus, can be formed from monomers such as glycolic acid, lactic acid, propyl fumarate, caprolactone, hyaluronan, hyaluronic acid, and the like. Other lattices can include proteins, polysaccharides, polyhydroxy acids, polyorthoesthers, polyanhydrides, polyphosphazenes, or synthetic polymers (particularly biodegradable polymers). Of course, a suitable polymer for forming such lattice can include more than one monomer (e.g., combinations of the indicated monomers). Also, the lattice can also include hormones, such as growth factors, cytokines, and morphogens (e.g., retinoic acid, aracadonic acid, and the like), desired extracellular matrix molecules (e.g., polyornithine, fibronectin, laminin, collagen, and the like), or other materials (i.e., DNA, viruses, other cell types, and the like) as desired.

In another embodiment, the invention provides a lattice composition comprising NSCs of the present invention and mature/differentiated cells of a desired phenotype thereof, particularly to increase the induction of the NSCs to differentiate appropriately within the lattice (i.e., as an effect of co-culturing such cells within the lattice).

The low oxygen condition of the present invention can be used to culture any NSC, for example short term and long term proliferation of NSCs. The NSCs can be derived from any source including but not limited to mouse, rat, and human. In addition, NSCs and their differentiated progeny may be immortalized or conditionally immortalized using techniques known in the art. Alternatively, the NSCs can be used as primary cultures, whereby the cells have not been cultured in a manner that would transform or immortalize the NSCs.

Characterization

At any time point during the culturing of the cells under low oxygen conditions, the cells can be harvested and collected for immediate experimental/therapeutic use or cryopreserved for use at a later time. NSCs described herein may be cryopreserved according to routine procedures. Preferably, about one to ten million cells are cryopreserved in NSC medium with 10% DMSO in vapor phase of Liquid N₂. Frozen cells can be thawed by swirling in a 37° C. bath, resuspended in fresh proliferation medium, and grown as usual. Cryopreservation is a procedure common in the art and as used herein encompasses all procedures currently used to cryopreserve cells for future analysis and use.

In another aspect, the cells can be harvested and subjected to flow cytometry to evaluate cell surface markers to assess the change in phenotype of the cells in view of the culture conditions.

NSCs cells may be characterized using any one of numerous methods in the art and methods disclosed herein. The cells may be characterized by the identification of surface and intracellular proteins, genes, and/or other markers indicative of differentiation of the cells such that they express at least one characteristic of a differentiated cell, such as a neuron. These methods include, but are not limited to, (a) detection of cell surface proteins by immunofluorescent assays such as flow cytometry or in situ immunostaining of cell surface proteins such as O4, CD45, CD 56, CD86, CD14, CD133, CD184, CD80, CD34, MHC class II molecules and MHC class I molecules; (b) detection of intracellular proteins such as nestin, MAP2, GFAP by immunofluorescent methods such as flow cytometry or in situ immunostaining using specific antibodies; (c) detection of the expression mRNAs by methods such as polymerase chain reaction, in situ hybridization, and/or other blot analysis.

Phenotypic markers of the desired cell are well known to those of ordinary skill in the art. Lineage specific phenotypic characteristics can include cell surface proteins, cytoskeletal proteins, cell morphology, and secretory products. As discussed elsewhere herein, NSCs can be differentiated to express a protein marker specific for a neuron, a glial cell, an astroycte or an oligodendrocyte.

In order to identify the cellular phenotype either during proliferation or differentiation of the NSCs, various cell surface or intracellular markers may be used. When the NSCs of the invention are proliferating, anti-nestin antibody can be used as a marker to identify undifferentiated cells.

When differentiated, most of the NSCs lose their nestin positive immunoreactivity. In particular, antibodies specific for various neuronal or glial proteins may be employed to identify the phenotypic properties of the differentiated NSCs. Neurons may be identified using antibodies to neuron specific enolase (“NSE”), neurofilament, tau, β-tubulin, or other known neuronal markers. Astrocytes may be identified using antibodies to glial fibrillary acidic protein (“GFAP”), or other known astrocytic markers. Oligodendrocytes may be identified using antibodies to galactocerebroside, O4, myelin basic protein (“MBP”) or other known oligodendrocytic markers.

It is also possible to identify cell phenotypes by identifying compounds characteristically produced by those phenotypes. For example, it is possible to identify neurons by their ability to produce neurotransmitters such as acetylcholine, dopamine, epinephrine, norepinephrine, and the like.

Specific neuronal phenotypes can be identified according to the specific products produced by those neurons. For example, GABA-ergic neurons may be identified by the production of glutamic acid decarboxylase (“GAD”) or GABA. Dopaminergic neurons may be identified by the production of dopa decarboxylase (“DDC”), dopamine or tyrosine hydroxylase (“TH”). Cholinergic neurons may be identified by the production of choline acetyltransferase (“ChAT”). Hippocampal neurons may be identified by staining with NeuN. Based on the present disclosure, one skilled in the art would appreciate that any suitable known marker for identifying specific neuronal phenotypes may be used.

In addition to characterizing the cells using neuronal markers, the cells can be genetically analyzed using methods discussed elsewhere herein including but not limited to SNP (single nucleotide polymorphism) genotyping, HLA (human leukocyte antigen) typing, karyotyping, DNA fingerprinting and genomic stability tests.

Methods of using NSCs

The invention provides a differentiated cell population containing neurons, as well as astrocytes and oligodendrocytes. Typically, using methods in the art, NSC cultures form very few neurons. According to the methods disclosed herein, a larger number of neurons can be obtained because a larger number of NSCs can be generated under low oxygen conditions. Thus, the methods of the present invention are advantageous as they facilitate the generation of a larger amount of a neuronal population prior to implantation into a patient having a disorder or disease of the CNS where transplantation of differentiated cells are desired.

The present invention also relates to the discovery that the expression of Nestin molecules by NSCs can be modulated by culturing NSCs under low oxygen conditions. The present invention provides a method of culturing NSCs in a manner that provides additional benefits over the standard methods used for culturing NSCs. These benefits include, but are not limited to enhancing the proliferation of the NSCs while maintaining the multipotential capacities of the NSCs and increasing Nestin molecule expression by the NSCs. Preferably, the cells are cultured under low oxygen conditions to generate a population of cells suitable for therapeutic use.

NSCs obtained by methods of the present invention can be induced to differentiate into neurons, astrocytes, oligodendrocytes and the like by selection of culture conditions known in the art to lead to differentiation of NSCs into cells of a selected type.

NSCs cultured or expanded as described in this disclosure can be used to treat a variety of disorders known in the art to be treatable using NSCs. The NSCs are useful in these treatment methods can include those that have, and those that do not have an exogenous gene inserted therein. Examples of such disorders include but are not limited to brain trauma, Huntington's disease, Alzheimer's disease, Parkinson's disease, spinal cord injury, stroke, multiple sclerosis, cancer, CNS lysosomal storage diseases and head trauma.

The NSCs of the present invention described herein, and their differentiated progeny may be immortalized or conditionally immortalized using known techniques. Alternatively, the NSCs can be used as a primary culture, whereby the cells have not been cultured in a manner that would transform or immortalize the NSCs.

The NSCs of this invention have numerous uses, including for drug screening, diagnostics, genomics and transplantation. The cells of the present invention can be induced to differentiate into the neural cell type of choice using the appropriate media described in this invention. The drug to be tested can be added prior to differentiation to test for developmental inhibition, or added post-differentiation to monitor neural cell-type specific reactions.

Genetic Modification

The cells of the present invention can also be used to express a foreign protein or molecule for a therapeutic purpose or for a method of tracking their integration and differentiation in a patient's tissue. Thus, the invention encompasses expression vectors and methods for the introduction of exogenous DNA into the cells with concomitant expression of the exogenous DNA in the cells such as those described, for example, in Sambrook et al. (2002, Molecular Cloning: A Laboratory Manual, Cold Spring Harbor Laboratory, New York), and in Ausubel et al. (1997, Current Protocols in Molecular Biology, John Wiley & Sons, New York).

The isolated nucleic acid can encode a molecule used to track the migration, integration, and survival of NSCs once they are placed in the patient, or they can be used to express a protein that is mutated, deficient, or otherwise dysfunctional in the patient. Proteins for tracking can include, but are not limited to green fluorescent protein (GFP), any of the other fluorescent proteins (i.e., enhanced green, cyan, yellow, blue and red fluorescent proteins; Clontech, Palo Alto, Calif.), or other tag proteins (i.e., LacZ, FLAG-tag, Myc, His₆, and the like) disclosed elsewhere herein.

The present invention is also useful for obtaining NSCs that express an exogenous gene, so that the NSCs can be used, for example, for cell therapy or gene therapy. That is, the present invention allows for the production of large numbers of NSCs which express an exogenous gene. The exogenous gene can, for example, be an exogenous version of an endogenous gene (i.e., a wild type version of the same gene can be used to replace a defective allele comprising a mutation). The exogenous gene is usually, but not necessarily, covalently linked with (i.e., “fused with”) one or more additional genes. Exemplary “additional” genes include a gene used for “positive” selection to select cells that have incorporated the exogenous gene, and a gene used for “negative” selection to select cells that have incorporated the exogenous gene into the same chromosomal locus as the endogenous gene or both.

An NSC expressing a desired exogenous can be used to provide the product of the exogenous gene to a cell, tissue, or whole mammal where a higher level of the gene product can be useful to treat or alleviate a disease, disorder or condition associated with abnormal expression, and/or activity. Therefore, the invention includes an NSC expressing an exogenous gene where increasing expression, protein level, and/or activity of the desired gene product can be useful to treat or alleviate a disease, disorder or condition.

When the purpose of genetic modification of the cell is for the production of a biologically active substance, the substance will generally be one that is useful for the treatment of a given CNS disorder. For example, it may be desired to genetically modify cells so that they secrete a certain growth factor product.

The cells of the present invention can be genetically modified by having exogenous genetic material introduced into the cells, to produce a molecule such as a trophic factor, a growth factor, a cytokine, a neurotrophin, and the like, which is beneficial to culturing the cells. In addition, by having the cells genetically modified to produce such a molecule, the cell can provide an additional therapeutic effect to the patient when transplanted into a patient in need thereof.

As used herein, the term “growth factor product” refers to a protein, peptide, mitogen, or other molecule having a growth, proliferative, differentiative, or trophic effect on a cell. Growth factor products useful in the treatment of CNS disorders include, but are not limited to, nerve growth factor (NGF), brain-derived neurotrophic factor (BDNF), the neurotrophins (NT-3, NT-4/NT-5), ciliary neurotrophic factor (CNTF), amphiregulin, FGF-1, FGF-2, EGF, TGFα, TGFβs, PDGF, IGFs, and the interleukins; IL-2, IL-12, IL-13.

Cells can also be modified to express a certain growth factor receptor (r) including, but not limited to, p75 low affinity NGFr, CNTFr, the trk family of neurotrophin receptors (trk, trkB, trkC), EGFr, FGFr, and amphiregulin receptors. Cells can be engineered to produce various neurotransmitters or their receptors such as serotonin, L-dopa, dopamine, norepinephrine, epinephrine, tachykinin, substance-P, endorphin, enkephalin, histamine, N-methyl D-aspartate, glycine, glutamate, GABA, ACh, and the like. Useful neurotransmitter-synthesizing genes include TH, dopa-decarboxylase (DDC), DBH, PNMT, GAD, tryptophan hydroxylase, ChAT, and histidine decarboxylase. Genes that encode various neuropeptides which may prove useful in the treatment of CNS disorders, include substance-P, neuropeptide-Y, enkephalin, vasopressin, VIP, glucagon, bombesin, cholecystokinin (CCK), somatostatin, calcitonin gene-related peptide, and the like.

According to the present invention, gene constructs which comprise nucleotide sequences that encode heterologous proteins are introduced into the NSCs. That is, the cells are genetically modified to introduce a gene whose expression has therapeutic effect in the individual. According to some aspects of the invention, NSCs from the individual to be treated or from another individual, or from a non-human animal, may be genetically modified to replace a defective gene and/or to introduce a gene whose expression has therapeutic effect in the individual being treated.

The term “genetic modification” as used herein refers to the stable or transient alteration of the genotype of an NSC by intentional introduction of exogenous DNA. DNA may be synthetic, or naturally derived, and may contain genes, portions of genes, or other useful DNA sequences. The term “genetic modification” as used herein is not meant to include naturally occurring alterations such as that which occurs through natural viral activity, natural genetic recombination, or the like.

Exogenous DNA may be introduced to an NSC using viral vectors (retrovirus, modified herpes viral, herpes-viral, adenovirus, adeno-associated virus, lentiviral, and the like) or by direct DNA transfection (lipofection, calcium phosphate transfection, DEAE-dextran, electroporation, and the like). The genetically modified cells of the present invention possess the added advantage of having the capacity to fully differentiate to produce neurons or differentiated cells in a reproducible fashion using a number of differentiation protocols.

In all cases in which a gene construct is transfected into a cell, the heterologous gene is operably linked to regulatory sequences required to achieve expression of the gene in the cell. Such regulatory sequences typically include a promoter and a polyadenylation signal.

The gene construct is preferably provided as an expression vector that includes the coding sequence for a heterologous protein operably linked to essential regulatory sequences such that when the vector is transfected into the cell, the coding sequence will be expressed by the cell. The coding sequence is operably linked to the regulatory elements necessary for expression of that sequence in the cells. The nucleotide sequence that encodes the protein may be cDNA, genomic DNA, synthesized DNA or a hybrid thereof or an RNA molecule such as mRNA.

The gene construct includes the nucleotide sequence encoding the beneficial protein operably linked to the regulatory elements and may remain present in the cell as a functioning cytoplasmic molecule, a functioning episomal molecule or it may integrate into the cell's chromosomal DNA. Exogenous genetic material may be introduced into cells where it remains as separate genetic material in the form of a plasmid. Alternatively, linear DNA which can integrate into the chromosome may be introduced into the cell. When introducing DNA into the cell, reagents which promote DNA integration into chromosomes may be added. DNA sequences which are useful to promote integration may also be included in the DNA molecule. Alternatively, RNA may be introduced into the cell.

The regulatory elements for gene expression include: a promoter, an initiation codon, a stop codon, and a polyadenylation signal. It is preferred that these elements be operable in the cells of the present invention. Moreover, it is preferred that these elements be operably linked to the nucleotide sequence that encodes the protein such that the nucleotide sequence can be expressed in the cells and thus the protein can be produced. Initiation codons and stop codons are generally considered to be part of a nucleotide sequence that encodes the protein. However, it is preferred that these elements are functional in the cells. Similarly, promoters and polyadenylation signals used must be functional within the cells of the present invention. Examples of promoters useful to practice the present invention include but are not limited to promoters that are active in many cells such as the cytomegalovirus promoter, SV40 promoters and retroviral promoters. Other examples of promoters useful to practice the present invention include but are not limited to tissue-specific promoters, i.e. promoters that function in some tissues but not in others; also, promoters of genes normally expressed in the cells with or without specific or general enhancer sequences. In some embodiments, promoters are used which constitutively express genes in the cells with or without enhancer sequences. Enhancer sequences are provided in such embodiments when appropriate or desirable.

The cells of the present invention can be transfected using well known techniques readily available to those having ordinary skill in the art. Exogenous genes may be introduced into the cells using standard methods where the cell expresses the protein encoded by the gene. In some embodiments, cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some embodiments, recombinant viral vectors are used to introduce DNA with desired sequences into the cell. In some embodiments, recombinant retrovirus vectors are used to introduce DNA with desired sequences into the cells. In some embodiments, standard CaPO₄, DEAE dextran or lipid carrier mediated transfection techniques are employed to incorporate desired DNA into dividing cells. Standard antibiotic resistance selection techniques can be used to identify and select transfected cells. In some embodiments, DNA is introduced directly into cells by microinjection. Similarly, well-known electroporation or particle bombardment techniques can be used to introduce foreign DNA into the cells. A second gene is usually co-transfected or linked to the therapeutic gene. The second gene is frequently a selectable antibiotic-resistance gene. Transfected cells can be selected by growing the cells in an antibiotic that will kill cells that do not take up the selectable gene. In most cases where the two genes are unlinked and co-transfected, the cells that survive the antibiotic treatment have both genes in them and express both of them.

Use of Isolated Neural Stem Cells

Isolated neural stem cells are useful in a variety of ways. These cells can be used to reconstitute cells in a mammal whose cells have been lost through disease or injury. Genetic diseases may be treated by genetic modification of autologous or allogeneic neural stem cells to correct a genetic defect or to protect against disease. Diseases related to the lack of a particular secreted product such as a hormone, an enzyme, a growth factor, or the like may also be treated using NSCs. CNS disorders encompass numerous afflictions such as neurodegenerative diseases (i.e. Alzheimer's and Parkinson's), acute brain injury (i.e. stroke, head injury, cerebral palsy) and a large number of CNS dysfunctions (i.e. depression, epilepsy, and schizophrenia). Diseases including but are not limited to Alzheimer's disease, multiple sclerosis (MS), Huntington's Chorea, amyotrophic lateral sclerosis (ALS), and Parkinson's disease, have all been linked to the degeneration of neural cells in particular locations of the CNS, leading to the inability of these cells or the brain region to carry out their intended function. NSCs isolated and cultured as described herein can be used as a source of progenitor cells and committed cells to treat these diseases.

The NSCs cultured as described herein may be frozen at liquid nitrogen temperatures and stored for long periods of time, after which they can be thawed and are capable of being reused. The cells are usually stored in 10% DMSO and 90% complete growth medium. Once thawed, the cells may be expanded using the methods described elsewhere herein.

NSCs obtained using the methods of the present invention can be induced to differentiate into neurons, astrocytes, oligodendrocytes and the like by selection of culture conditions known in the art to lead to differentiation of NSCs into cells of a selected type. For example, NSCs can be induced to differentiate by plating the cells on a coated surface, preferably polyornithine or poly-L-lysine (PPL), in the absence of growth factors but in the presence of 10% fetal bovine serum (FBS). Differentiation can also be induced by plating the cells on a fixed substrate such as flasks, plates, or coverslips coated with an ionically charged surface such as poly-L-lysine and poly-L-ornithine and the like. Other substrates may be used to induce differentiation such as collagen, fibronectin, laminin, MATRIGEL™ (Collaborative Research), and the like.

A preferred method for inducing differentiation of the neural stem cell progeny comprises culturing the cells on a fixed substrate in a culture medium that is free of proliferation-inducing growth factor. After removal of the proliferation-inducing growth factor, the cells adhere to the substrate (i.e. poly-ornithine-treated plastic or glass), flatten, and begin to differentiate into neurons and glial cells. At this stage, the culture medium may contain serum such as 0.5-1.0% fetal bovine serum (FBS). However, for certain uses, if defined conditions are required, serum should not be used. Within 2-3 days, most or all of the neural stem cell progeny begin to lose immunoreactivity for nestin and begin to express antigens specific for neurons, astrocytes or oligodendrocytes as determined by immunocytochemistry techniques well known in the art. In particular, cellular markers for neurons include but not limited to neuron-specific enolase (NSE), neurofilament (NF), β-tubulin, MAP-2; and for glial, GFAP, galactocerebroside (GalC) (a myelin glycolipid identifier of oligodendrocytes), and the like.

NSCs cultured or expanded as described in this disclosure can be used, as cultured, or they can be used following differentiation into selected cell types, to treat a variety of disorders known in the art to be treatable using NSCs. The NSCs that are useful in these treatment methods include those that have, and those that do not have an exogenous gene inserted therein. Examples of disorders that can be treated include but are not limited to brain trauma, Huntington's Chorea, Alzheimer's disease, Parkinson's disease, spinal cord injury, stroke, multiple sclerosis, head trauma and other such diseases and/or injuries where the replacement of tissue by the cells of the present invention can result in a treatment or alleviation of the disease and/or injuries.

Transplantation

Laboratory and clinical studies have shown the transplantation of cells into the CNS is a potentially significant alternative therapeutic modality for neurodegenerative disorders such as Parkinson's disease (Wictorin et al., 1992, J Comp Neurol. 323:475-94; Lindvall et al., 1990, Science 247:574-7; Bjorklund and Stenevi, 1984, Annu Rev Neurosci. 7:279-308). In some cases, transplanted neural tissue can survive and form connections with the CNS of the recipient (e.g. a host) (Wictorin et al., 1992, J Comp Neurol. 323:475-94). When successfully accepted by the host, the transplanted cells and/or tissue have been shown to ameliorate the behavioral deficits associated with the disorder. The obligatory step for the success of this kind of treatment is to have enough viable cells available for the transplant. The low oxygen culturing conditions described herein can be used to culture and differentiate NSCs for transplantation.

Fetal neural tissue is another important source for neural transplantation (Lindvall et al., 1990, Science 247:574-7; Bjorklund, 1992, Curr Opin Neurobiol. 2:683-9; Isacson et al., 1995, Nat. Med. 1: 1189-94). Other viable graft sources include adrenal cells and various cell types that secrete neural growth factors and trophic factors. The field of neural tissue transplantation as a productive treatment protocol for neurodegenerative disorders has received much attention resulting in its progression to clinical trials. The present invention provides a method of maintaining such tissue in a state that prevents them from losing their ability to serve as an appropriate graft for neurodegenerative diseases.

Methods of grafting cells are now well known to those of skill in art (U.S. Pat. Nos. 5,762,926; 5,650,148; 5,082,670). Neural transplantation or grafting involves transplantation of cells into the central nervous system or into the ventricular cavities or subdurally onto the surface of a host brain. Conditions for successful transplantation include: 1) viability of the implant; 2) retention of the graft at the site of transplantation; and 3) minimum amount of pathological reaction at the site of transplantation.

The present invention encompasses methods for administering the cells of the present invention to an animal, including humans, in order to treat diseases where the introduction of new, undamaged cells will provide some form of therapeutic relief.

The cells of the present invention can be administered as an NSC or an NSC that has been induced to differentiate to exhibit at least one characteristic of a neuronal like cell. The skilled artisan will readily understand that NSCs can be administered to a recipient as a differentiated cell, for example, a neuron, and is useful in replacing diseased or damaged neurons in the animal. Additionally, NSCs can be administered as an undifferentiated cell and upon receiving signals and cues from the surrounding milieu, can differentiate into a desired cell type dictated by the neighboring cellular milieu.

The cells can be prepared for grafting to ensure long term survival in the in vivo environment. For example, cells are propagated in a suitable culture medium for growth and maintenance of the cells and are allowed to grow to confluency. The cells are loosened from the culture substrate using, for example, a buffered solution such as phosphate buffered saline (PBS) containing 0.05% trypsin supplemented with 1 mg/ml of glucose; 0.1 mg/ml of MgCl₂, 0.1 mg/ml CaCl₂ (complete PBS) plus soybean trypsin inhibitor to inactivate trypsin. The cells can be washed with PBS and are then resuspended in the complete PBS without trypsin and at a selected density for injection.

In addition to PBS, any osmotically balanced solution which is physiologically compatible with the host subject may be used to suspend and inject the donor cells into the host. Formulations of a pharmaceutical composition suitable for parenteral administration comprise the active ingredient, i.e. the cells, combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The invention also encompasses grafting NSCs (or differentiated NSCs) in combination with other therapeutic procedures to treat a disease or trauma to the CNS and peripheral regions. Thus, the cells of the invention may be co-grafted with other cells, both genetically modified or non-genetically modified cells which exert beneficial effects on the patient. Therefore the methods disclosed herein can be combined with other therapeutic procedures as would be understood by one skilled in the art once armed with the teachings provided herein.

The cells can be transplanted as a mixture/solution comprising of single cells or a solution comprising a suspension of a cell aggregate. Such aggregates can be approximately 10-500 micrometers in diameter, more preferably, about 40-50 micrometers in diameter. A cell aggregate can comprise about 5-100 cells per sphere, more preferably, about 5-20, cells per sphere. The density of transplanted cells can range from about 10,000 to 1,000,000 cells per microliter, more preferably, from about 25,000 to 500,000 cells per microliter.

The mode of administration of the cells of the invention to the CNS of the mammal may vary depending on several factors including the type of disease being treated, the age of the mammal, whether the cells are differentiated or not, whether the cells have heterologous DNA introduced therein, and the like. Cells may be introduced to the desired site by direct injection, or by any other means used in the art for the introduction of compounds into the CNS.

The cells can be administered into a host in a wide variety of ways. Modes of administration include, but are not limited to, intravascular, intracerebral, parenteral, intraperitoneal, intravenous, epidural, intraspinal, intrasternal, intra-articular, intra-synovial, intrathecal, intra-arterial, intracardiac, or intramuscular.

Transplantation of the cells of the present invention can be accomplished using techniques well known in the art as well as those described herein or as developed in the future. The present invention comprises a method for transplanting, grafting, infusing, or otherwise introducing NSCs or differentiated NSCs into a mammal, preferably, a human. Exemplified below are methods for transplanting the cells into the brains of both rodents and humans, but the present invention is not limited to such anatomical sites or to those animals. Also, methods for bone transplants are well known in the art and are described in, for example, U.S. Pat. No. 4,678,470, pancreas cell transplants are described in U.S. Pat. No. 6,342,479, and U.S. Pat. No. 5,571,083, teaches methods for transplanting cells, such as NSCs, to any anatomical location in the body.

In order to transplant the cells of the present invention into a human, the cells are prepared as described herein. Preferably, the cells are from the patient for which the cells are being transplanted into (autologous transplantation). One preferable mode of administration is as follows. In the case where cells are not from the patient (allogeneic transplantation), at a minimum, blood type or haplotype compatibility should be determined between the donor cell and the patient. Surgery is performed using a Brown-Roberts-Wells computed tomographic (CT) stereotaxic guide. The patient is given local anesthesia in the scalp area and intravenously administered midazolam. The patient undergoes CT scanning to establish the coordinates of the region to receive the transplant. The injection cannula usually consists of a 17-gauge stainless steel outer cannula with a 19-gauge inner stylet. This is inserted into the brain to the correct coordinates, then removed and replaced with a 19-gauge infusion cannula that has been preloaded with about 30 μl of tissue suspension. The cells are slowly infused at a rate of about 3 μl/min as the cannula is withdrawn. Multiple stereotactic needle passes are made throughout the area of interest, approximately 4 mm apart. The patient is examined by CT scan postoperatively for hemorrhage or edema. Neurological evaluations are performed at various post-operative intervals, as well as PET scans to determine metabolic activity of the implanted cells.

Between about 10⁵ and about 10¹³ cells per 100 kg person are administered to a human per infusion. In some embodiments, between about 1.5×10⁶ and about 1.5×10¹² cells are infused per 100 kg person. In some embodiments, between about 1×10⁹ and about 5×10¹¹ cells are infused per 100 kg person. In some embodiments, between about 4×10⁹ and about 2×10¹¹ cells are infused per 100 kg person. In other embodiments, between about 5×10⁸ cells and about 1×10¹ cells are infused per 100 kg person.

In some embodiments, a single administration of cells is provided. In some embodiments, multiple administrations are provided. In some embodiments, multiple administrations are provided over the course of 3-7 consecutive days. In some embodiments, 3-7 administrations are provided over the course of 3-7 consecutive days. In other embodiments, 5 administrations are provided over the course of 5 consecutive days.

In some embodiments, a single administration of between about 10⁵ and about 10¹³ cells per 100 kg person is provided. In some embodiments, a single administration of between about 1.5×10⁸ and about 1.5×10¹² cells per 100 kg person is provided. In some embodiments, a single administration of between about 1×10⁹ and about 5×10¹¹ cells per 100 kg person is provided. In some embodiments, a single administration of about 5×10¹⁰ cells per 100 kg person is provided. In some embodiments, a single administration of 1×10¹⁰ cells per 100 kg person is provided.

In some embodiments, multiple administrations of between about 10⁵ and about 10¹³ cells per 100 kg person are provided. In some embodiments, multiple administrations of between about 1.5×10⁸ and about 1.5×10¹² cells per 100 kg person are provided. In some embodiments, multiple administrations of between about 1×10⁹ and about 5×10¹¹ cells per 100 kg person are provided over the course of 3-7 consecutive days. In some embodiments, multiple administrations of about 4×10⁹ cells per 100 kg person are provided over the course of 3-7 consecutive days. In some embodiments, multiple administrations of about 2×10¹¹ cells per 100 kg person are provided over the course of 3-7 consecutive days. In some embodiments, 5 administrations of about 3.5×10⁹ cells are provided over the course of 5 consecutive days. In some embodiments, 5 administrations of about 4×10⁹ cells are provided over the course of 5 consecutive days. In some embodiments, 5 administrations of about 1.3×10¹¹ cells are provided over the course of 5 consecutive days. In some embodiments, 5 administrations of about 2×10¹¹ cells are provided over the course of 5 consecutive days.

In one embodiment of the invention, the cells of the present invention are administered to a mammal suffering from a disease, disorder or condition involving the CNS, in order to augment or replace the diseased and damaged cells of the CNS. NSCs are preferably administered to a human suffering from a disease, disorder or condition involving the CNS. The NSCs are further preferably administered to the brain or spinal cord of the human. In some instances, the cells are administered to the adjacent site of injury in the human brain. The precise site of administration of the cells depends on any number of factors, including but not limited to, the site of the lesion to be treated, the type of disease being treated, the age of the human and the severity of the disease, and the like. Determination of the site of administration is well within the skill of the artisan versed in the administration of such cells.

The following examples further illustrate aspects of the present invention. However, they are in no way a limitation of the teachings or disclosure of the present invention as set forth herein.

### EXAMPLES

The invention is now described with reference to the following Examples. These Examples are provided for the purpose of illustration only, and the invention is not limited to these Examples, but rather encompasses all variations which are evident as a result of the teachings provided herein.

The following experiments demonstrate that that expansion of NSCs under more physiological oxygen than atmospheric oxygen increases their expansion rate while maintaining their multipotency. The following experiments also address the effect of lower oxygen on expression of stem cell or progenitor cell markers, senescence/apoptosis, and differentiation of NSCs.

NSCs cultured according to the methods described herein demonstrates a feasible means for using NSCs in cell and/or gene therapy and provides support for the clinical use of NSCs as an “off the shelf” product.

### Example 1

### Isolation and Culturing of Neural Stem Cells

Human fetal brain tissue was purchased from Advanced Bioscience Resources (Alameda, Calif.). The tissue was washed with phosphate buffered saline (PBS) supplemented with penicillin/streptomycin solution. The tissue was then placed in a sterile Petri dish in cold PBS supplemented with penicillin/streptomycin to further clean the tissue and remove the menninges. The tissue was teased with a pair of forceps to break the tissue into smaller pieces. The tissue was dissociated using a Pasteur pipette (about 20 times) to triturate the tissue. The tissue was further dissociated using a Pasteur pipette fire-polished to significantly reduce the bore size (20 times) to triturate the tissue.

The resulting cells were pelleted by centrifugation at 1000 r.p.m. for 5 minutes at room temperature. The cell pellet was resuspended in 10 ml of growth medium (DMEM/F12 (Invitrogen), 8 mM glucose, glutamine, 20 mM sodium bicarbonate, 15 mM HEPES, 8 μg/ml Heparin (Sigma), N2 supplement (Invitrogen), 10 ng/ml bFGF (Peprotech), 20 ng/ml EGF (Peprotech)). The cells were plated on a coated T-25 cm² flask with vented cap and grown in a 5% CO₂ incubator at 37° C. Cultures were fed every other day by replacing 50% of the medium with fresh complete growth medium.

To passage the cells, the cells were trypsinized using 0.05% trypsin-EDTA in PBS for 2-3 minutes followed by addition of soybean trypsin inhibitor to inactivate the trypsin. The cells were pelleted at 1200 r.p.m. for 5 minutes at room temperature and then were resuspended in growth medium. Cells were plated at 100,000-125,000 cells/cm² on coated flasks. Cells were cryopreserved in 10% DMSO+90% complete growth medium.

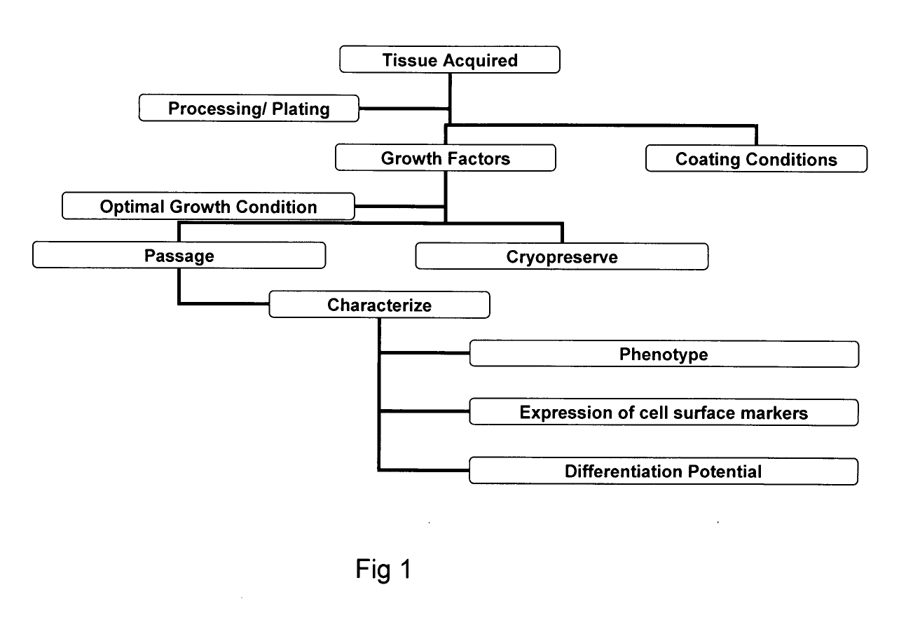

Several different human fetal brain and spinal cord derived cultures were established, expanded and characterized according to the flow chart of FIG. 1. For example, the human fetal brain stem cell culture designated THD-hWB-015 was established, expanded and cryopreserved as described above. THD-hWB-015 was maintained on coated flasks in DMEM/F12, 8 mM glucose, glutamine, 20 mM sodium bicarbonate, 15 mM HEPES, 8 μg/ml Heparin, N2 supplement, 10 ng/ml bFGF, 20 ng/ml EGF.

To coat a flask, 15 μg/ml polyornithine (Sigma) in 1×PBS was added to the flask and the flask was incubated overnight at 37° C. in an incubator. Excess polyornithine was removed from the flask the next day. The flask was washed three times with 1×PBS and 10 μg/ml human fibronectin (Chemicon) in 1×PBS was added to the flask, and the flask was incubated for at least 4 hrs at 37° C. Before using the “coated” flask to culture the cells of the present invention, excess fibronectin was removed from the flask.

THD-hWB-015 was cultured under low oxygen conditions and tested for their proliferation and multipotential to differentiate into different brain cell lineages. Cells from this culture were thawed at passage 7 and then grown for six consecutive passages under four different growth conditions to assess their proliferation rate.

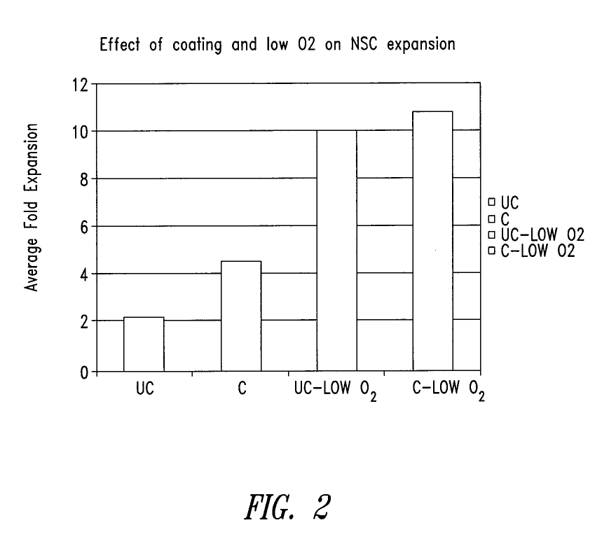

A positive effect on cell proliferation was observed when cells were grown under lower oxygen (3-6% O₂). Cells grew at least two-three fold better than cells grown under oxygen (about 20 oxygen) levels. The highest proliferation was observed when the cells were grown on coated dishes under lower oxygen (FIG. 2).

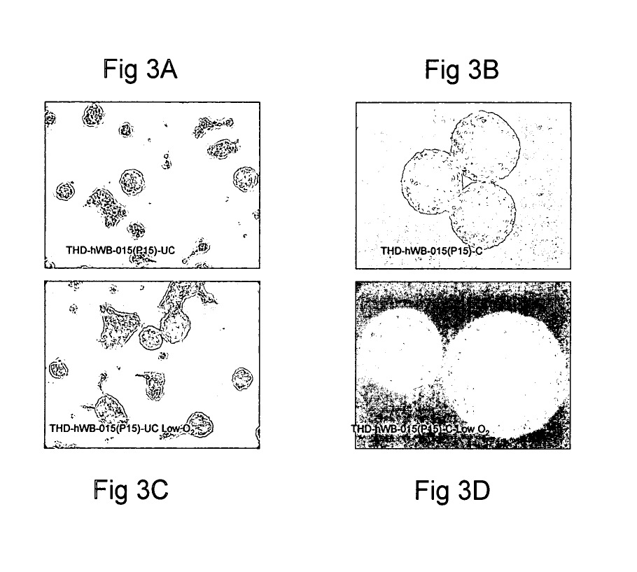

There were some differences in the morphology of cells cultured under different growth conditions. NSCs cultured under lower oxygen formed larger neurospheres (FIG. 3). The differences in neurosphere sizes from these studies corresponded with the differences in the expansion rate. A preferred NSC growth condition based on this data is coating the plates and growing the cells in reduced oxygen. It was observed that at every passage, NSCs grew better under reduced oxygen and formed larger neurospheres.

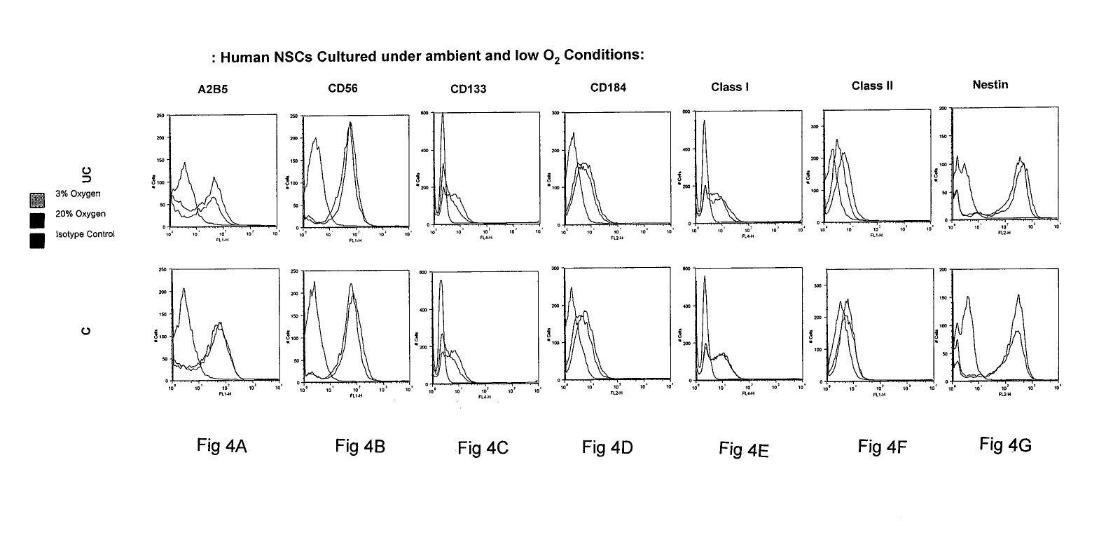

To characterize NSCs that were cultured for six consecutive passages in different growth conditions, a number of different markers previously shown to be expressed by these cells were analyzed (Table 1, see below) by flow cytometry. Cell surface markers known to be expressed on NSCs (CD133), on glial progenitors (A2B5), on neural progenitors (CD56), on migratory cells (CD 184) and markers important for immunology (MHC class I and II) as well as nestin were selected (FIG. 4).

TABLE 1

| Positive | Weakly Positive | Negative |
|---|---|---|
| A2B5, CD29, CD44, | ABCG2, CD63, | CD11a, CD13, CD14, |
| CD54, CD56, CD90, | CD73, CD80, | CD31, CD34, CD40, CD45, |
| CD133, CD146, | CD166, HLA-DR | CD49a, CD86, CD105 |
| MHC class I | | |

Positive = 50-95%, Weakly Positive = 10-25% and Negative = 3% or less percent cells are positive for the marker.

Flow cytometric analyses of human fetal neural stem cells (THD-hWB-015) grown under four different growth conditions in the presence of bFGF and EGF for 14 days were performed. Cells were harvested and FACS analysis was carried out on approximately 2×10⁶ cells. The NSC populations were analyzed for surface expression of the following antigens for phenotypic characterization: CD56, CD133, CD184 (Miltenyi Biotech), HLA-A,B,C and HLA-DR (BD-Pharmingen). Final analysis of expression was based on percent (+) event values relative to their respective isotype controls. The data are presented in FIG. 4 where the cell fluorescence intensity is depicted on the x-axis and the number of cellular events on the y-axis. Briefly, 10,000 total events were acquired on a Becton Dickinson FACSCaliber flow cytometer using Cell Quest acquisition software (BDIS) and the cells were analyzed using Flow Jo analysis software (Tree Star). As shown in FIG. 4, U=Uncoated, C=Coated, A2B5=glial progenitor, Nestin=stem cells/progenitors, CD56=NCAM, CD133=Prominin-1, CD184=CXCR4 and MHC class I and II=immunological markers.

Expression of CD133 increased slightly when NSCs isolated from brain tissue were grown under low oxygen conditions. There was no significant difference in the expression of this protein when NSCs isolated from spinal cord were cultured in low oxygen conditions. These studies demonstrated that under lower oxygen conditions, there was no significant difference in the expression of any of the markers tested. That being said, expression of CD133 increased slightly.

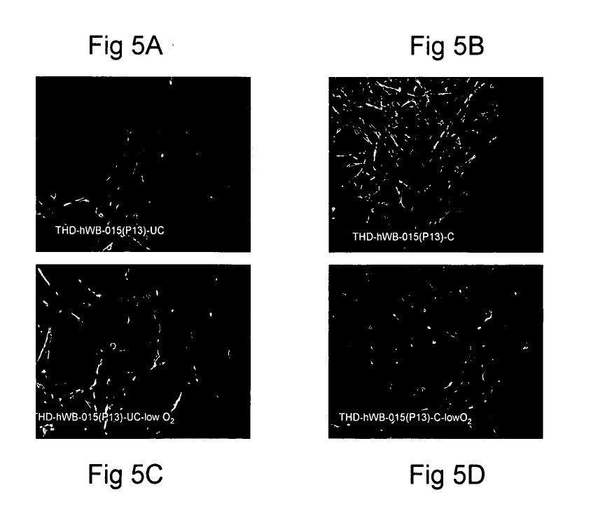

To assess whether NSCs were multipotent when cultured in low oxygen conditions, NSCs were subjected to differentiation conditions where they would differentiate into different brain cell lineages, such as neurons and astrocytes. 2×10⁵ cells were plated on coated chamber slides at passage 11 and 13. The NSCs were allowed to differentiate for 14 days by withdrawing the growth factors from the growth medium followed by further differentiating them in Neurobasal medium +GlutaMax +B27+10 ng/ml BDNF. The cells were fixed in 4% paraformaldehyde and stained with neuron specific anti-MAP2 and astrocytes specific anti-GFAP antibodies. Cell nuclei were stained with DAPI. Percentage of cells differentiating into neurons and astrocytes was assessed for each growth condition. NSCs grown under lower oxygen differentiated into 75-80% MAP2 positive neurons and 18-20% astrocytes. Under ambient oxygen, 65% of the cells differentiated into MAP2 positive neurons and 33% into astrocytes (FIG. 5).

In summary, the results presented herein demonstrate that NSCs from human fetal brain and spinal cord can be cultured under low oxygen levels. The cells have been maintained in culture for more than 20 passages. It was also observed that a preferred condition to grow the cells was on coated vessels under reduced oxygen. These cells were observed to differentiate into astrocytes and neurons in vitro. Cells grown under reduced oxygen differentiated into more neurons compared to NSCs cultured in 20% oxygen.

### Example 2

### Optimal Growth Conditions for In Vitro Expansion of Human Fetal Brain and Spinal Cord Derived NSCs Grown Under Atmospheric and Reduced Oxygen

The results presented herein demonstrated that NSCs derived from human fetal brain exhibited at least 2-3 fold higher expansion under lower oxygen (3-6% O₂) compared to ambient oxygen culture condition. The following experiments were designed to use these cells to repair damaged neural tissue, for example, injured spinal cord, and to generate a large enough quantity of these cells for clinical applications. Instead of using a range of lower oxygen, the following experiments used a more precise oxygen level of 3%, which is the physiological oxygen level in the human embryonic brain.

Previously grown and cryopreserved primary cultures (early passage, P3) of human NSCs from brain and spinal cord were plated on coated flasks in DMEM/F 12, 8 mM glucose, glutamine, 20 mM sodium bicarbonate, 15 mM HEPES, 8 μg/ml Heparin, N2 supplement (Invitrogen, Carlsbad, Calif.), 10 ng/ml bFGF, 20 ng/ml EGF (Peprotech, Rocky Hill, N.J.). The cells were cultured under different culture conditions (i) 20% O₂ and (ii) 3% O₂. The cells were cultured in a humidified incubator at 37° C. and 5% CO₂, 95% air (20% O₂) or in an incubator set at 5% CO₂ flushed with 10% CO2 plus 3% O2 plus 87% N2 to get approximately 3-5% O2 and 5% CO2. NSCs were fed with fresh medium every other day and passaged following 14 days in culture. Total number of live cells under each condition was counted by trypan blue exclusion assay. The total expansion number for each passage was also measured.

In order to determine total number of dividing cells under two different culture conditions, at every third passage, 20 μM BrdU is added to the cells after plating the cells for 7-9 days. After 24 hours, BrdU is removed and the cells are harvested. To harvest the non-adherent cells, the supernatant is centrifuged to pellet the cells and the adherent cells are trypsinized (typsin was neutralized using soybean trypsin inhibitor). The trypsinized cells are pelleted by centrifugation at 1200 rpm for 5 minutes at room temperature. The cells are then resuspend in growth medium by pipetting the cells several times. Live cells are counted by trypan blue exclusion assay using a hemocytometer. Incorporation of BrdU is measured using BD Biosciences BrdU-FLOW kit. The cells are fixed in BD Cytofix buffer for 15 minutes at room temperature and the cells are permeabilize in BD Cytoperm buffer for 10 minutes on ice. To expose the BrdU, the cells are incubated in DNase for 1 hour at 37° C. After washing, the DNase stains the BrdU antigen with fluorescently labled anti-BrdU antibodies for 20 minutes at room temperature. Unbound antibody is washed away and the cells are analyzed with a BD FACSCalibur flow cytometer. BrdU (thymidine analog) is incorporated into newly synthesized DNA when the cells enter and progress through the S phase of the cell cycle. The incorporated BrdU is stained with specific anti-BrdU fluorescent antibodies. The levels of cell-associated BrdU is measured by flow cytometry. This technique allowed for the identification of actively cycling (BrdU positive), as opposed to non-cycling (BrdU negative) cell fractions.

In order to determine the rate of cell division under two different culture conditions, a comparison of NSCs grown under lower O₂ and ambient O₂ can be made to determine if the higher rate of expansion is due to a shorter cell cycling time. A carboxyflurescein diacetate, succinimidyl ester (CFSE) washout method (Invitrogen, Carlsbad, Calif.) can be used according to manufacture's protocol to detect cell division.

CFSE passively diffuses into cells. Inside the cell, acetate groups from the dye are cleaved by intracellular esterases converting the dye into highly fluorescent ester. The ester group reacts with intracellular amines, forming fluorescent conjugates that are well retained even through cell division and can be measured by Flow cytometry. At every cell division, half the label is inherited by daughter cells.

NSCs are labeled with CFSE reagent for 15 minutes at 37° C. at every third passage. A washing step is employed to wash away excess unconjugated dye. The labeled cells are cultured for 1 day and 7 days under 3% and 20% oxygen before the cells are harvested. The amount of fluorescence is measured by flow cytometry. The rate of proliferation is measured based on shift in fluorescence due to cell division. Dividing cells exhibited an increased total number of cells and reduced fluorescence staining. The dye can be detected through several cell divisions.

The rate of apoptosis under the two different culture conditions can be measured using methods known in the art. A lower rate of apoptosis may account for the higher number of NSCs cultured under reduced oxygen conditions. Apoptosis is an evolutionarily conserved form of cell death which follows a specialized cellular process. The central component of this process is a cascade of proleolytic enzymes called caspases. In this assay, cell permeable, noncytotoxic, fluorescent-labeled caspase inhibitor binds covalently to the active caspase inside an apoptotic cell. Unbound inhibitor can be washed out and cells labeled with the fluorescent-labeled caspase inhibitor can be detected by fluorescent microscopy or flow cytometry. The cell nuclei can be stained with Hoechst, which allows for the calculation of total number of cells, percent apoptotic cells and percent non-apoptotic cells. This method is useful for the determination of whether there is reduced death in cells cultured under low oxygen conditions (e.g. 3% O₂).

Telomerase activity under the two different culture conditions can be measured. Telomeres are 6 base repeats found at the end of chromosomes in eukaryotes. In somatic cells, telomere length is progressively shortened with each cell division both in vivo and in vitro. Telomerase is a ribonucleoprotein that synthesizes and directs the telomeric repeats onto the 3′ end of existing telomeres. The telomerase activity can be detected by a sensitive PCR based method, TRAP (Telomeric Repeat Amplification Protocol). The TRAPeze XL kit developed by Chemicon International can be used according to the manufacture's protocol. This kit uses fluorescence energy transfer primers to generate fluorescently labeled TRAP products for quantitative analyses of telomerase activity. The fluorescence emission directly corresponds to telomerase activity. In this kit, Chemicon includes an internal control labeled with a second fluorophore to both monitor PCR amplification and aid in the quantification of telomerase activity. The effect of oxygen on telomerase activity in both human brain as well as spinal cord NSCs can be measured according to this method.

### Example 3

### Characterization of Brain and Spinal Cord NSCs Grown under Low Oxygen

To define the optimum growth condition for NSCs in vitro, factors such as the age of the tissue and the region from which the cells are derived should be considered. Another important aspect for maintaining any dividing cells in culture is the composition of medium and culture conditions. The composition of medium and culture condition controls both morphology and phenotype of cells in vitro. It has been demonstrated that Leukemia Inhibitory Factor (LIF) regulates specific sets of genes in NSCs. Based on the experiments presented herein, culturing NSCs in low oxygen conditions also regulates gene expression by NSCs. The effect of lower oxygen conditions on the fate of human NSCs derived from fetal brain was examined following the methods disclosed herein.

Expression of a number of cell surface markers on NSCs was examined by flow cytometry. The markers tested were selected based on their expression on NSC (neural stem cells), BMSC (bone marrow stem cells) or HSC (hematopoietic stem cells). Markers important for immunological activity were selected to predict if these cells would have an immune response upon transplantation or in mixed lymphocyte reaction in vitro.

The effect of growth conditions i.e. low versus ambient oxygen conditions on expression of these markers is a preliminary step to characterize these cells. These markers include neural stem cell specific markers (CD133), progenitor specific markers (CD56, A2B5, CD44), markers known to be expressed by bone marrow derived mesenchymal stem cells or hematopoietic stem cells (CD34, CD105) and markers important for immunology (MHC class I, HLA-DR, CD80, CD86). NSCs were grown under low or ambient oxygen conditions for 10 consecutive passages. At every third passage, the cells were harvested and analyzed as described elsewhere herein.

Expression of CD9 (oligodendrocyte progenitors) and other surface markers (Notch-1, syndecan-1, integrin-β1) shown to be expressed on neurosphere forming NSCs can also be measured. Expression of intracellular stem cell & progenitor specific markers (nestin, sox-2 and sox-1) can be measured by using quantitative PCR on RNA extracted from these cells.

Differentiation Potential

To determine the effect of low oxygen conditions on differentiation potential of NSCs, NSCs were differentiated (grown under different oxygen levels) for two weeks. 2×10⁵ cells were plated on coated chamber slides at every third passage. NSCs were allowed to differentiate for 14 days by withdrawing the growth factors from the growth medium followed by further differentiating them in Neurobasal medium +GlutaMax +B27+10 ng/ml BDNF or 10 ng/ml PDGF-AA (platelet derived growth factor) or motor neuron enhancing factors including, but not limited to NGF (nerve growth factor), CNTF (ciliary neurotrophic factor), Shh (sonic hedgehog), retinoic acid and the like. From these different differentiation regimens, the optimum condition for motor neuron or oligodendrocyte differentiation can be determined. The differentiation potential of these cells was measured by immunocytochemistry. NSCs differentiated under different conditions were stained with antibodies specific for neurons (Tuj1, MAP2), neuronal subtypes (γ-aminobutyric acid, tyrosine hydroxylase, peripherin, choline acetyltransferase), oligodendrocytes (O4, CNPase) and astrocytes (GFAP) to determine the differentiation of the cells. Cell nuclei were stained with DAPI. Human fetal brain and spinal cord derived cultures were used to study differentiation of NSCs under 3% O₂.

Total RNA was extracted from differentiated cells using Qiagen's RNeasy RNA extraction kit by following manufacturer's recommendation. Using quantitative PCR, the expression of glia specific gene (GFAP), neuron specific gene (MAP2), neuronal subtype specific genes like tyrosine hydroxylase (dopaminergic neurons), choline acetyltransferase (cholinergic neurons), peripherin (sensory and sympathetic neurons) as well as oligodendrocyte markers like OligI, CNPase was measured.

RealTime qRT-PCR assays for evaluating expression of human GFAP, MAP2, nestin, Musashi, Sox-2 and GAPDH has been successfully developed. Development of these assays involved using the Vector NTI software (v7.0; InforMax) to analyze the mRNA sequence of each gene and design primer pairs to amplify cDNA sequences in the range of 150-250 bp. Each amplified fragment was cloned into vector pCR2.1 (Invitrogen, Carlsbad, Calif.) according to the manufacturers instructions and the insert sequence was verified by Lark Technologies Inc. 500 ng of each unknown sample total RNA was compared against a standard curve generated using the sequence verified vector containing the gene fragment of interest (10⁵-10¹ copies) using Qiagen's QuantiTect SYBR Green RT-PCR kit according to the manufacturers instructions. All samples were run on a DNA Engine Opticon2 thermal cycler (MJ Research) with copies of specific product (based on melting curve profile) calculated from the standard curve by the Opticon Monitor Analysis software (v2.02). The standard curve lower limit of detection was gene-dependent, but was found to be at least 100 copies, with some cDNAs detectable at up to 10 copies. For each unknown sample, gene-specific copies were normalized to known copy numbers of GAPDH. The normalized number was then used as the basis of comparison between unknown samples (e.g. comparison of the expression of a particular gene between cell types or comparison between two treatment conditions for the same cell type). This assay was used to detect quantitative expression of the genes listed in Table 2.

TABLE 2

| | |
|---|---|
| Annealing | Product |
| Temp. | Size |

| | | | |
|---|---|---|---|
| Gene | Primers | (° C.) | (bp) |

| | | | |
|---|---|---|---|
| GFAP | S/5′-ACT CCC GAC CCG AGT GGA | 55 | 213 |
| | TT-3′; | | |
| | (SEQ ID NO:1) | | |
| | AS/5′-TAG ACG TCT GCC AGC TTG | | |
| | GTG G-3′; | | |
| | (SEQ ID NO:2) | | |
| MAP2 | S/5′-GTT CTA TCT CTT CTT CAG | 55 | 217 |
| | CAC GGC G-3′; | | |
| | (SEQ ID NO:3) | | |
| | AS/5′-CGG CAC CAA GAT GGC AGA | | |
| | CTT-3′; | | |
| | (SEQ ID NO:4) | | |
| Nestin | S/5′-CAG GGC AGC GTT GGA ACA | 57 | 214 |
| | GA-3′; | | |
| | (SEQ ID NO:5) | | |
| | AS/5′-TCT CAG CCT CCA GGA GGG | | |
| | TCC TGT A-3′; | | |
| | (SEQ ID NO:6) | | |
| Sox-2 | S/5′-CGT CAA GCG GCC CAT GAA | 55 | 156 |
| | TG-3′; | | |
| | (SEQ ID NO:7) | | |
| | AS/5′-TCG ATG AAC GGC CGC TTC | | |
| | TC-3′; | | |
| | (SEQ ID NO:8) | | |
| GAPDH | S/5′-CCC TGG CCA AGG TCA TCC | 60 | 238 |
| | ATG ACA-3′; | | |
| | (SEQ ID NO:9) | | |
| | AS/5′-AAA GGA GTG AGG CCC TGC | | |
| | AGC GTA-3′; | | |
| | (SEQ ID NO:10) | | |
| Tyrosine | S/5′-TCA TCA CCT GGT CAC CAA | 60 | 124 |
| hydroxylase | GTT-3′; | | |
| | (SEQ ID NO:11) | | |
| | AS/5′-GGT CGC CGT GCC TGT ACT- | | |
| | 3′; | | |
| | (SEQ ID NO:12) | | |
| Peripherin | S/5′-GCG CTC AAG CAG AGG TTG | 52 | 302 |
| | GA-3′; | | |
| | (SEQ ID NO:13) | | |
| | AS/5′-TTC GCG GCG ATG CTC TCG | | |
| | TA-3′; | | |
| | (SEQ ID NO:14) | | |
| CNPase | S/5′-GGA CAA GCC TGA GCT GCA | 51 | 274 |
| | TTT T-3′; | | |
| | (SEQ ID NO:15) | | |
| | AS/5′-AAT TCT GAT GTC CCG GCG | | |
| | GC-3′; | | |
| | SEQ ID NO:16 | | |
| Choline | S/5′-AGC ACT TCC CTG GCA CCG | 52 | 255 |
| acetyl- | AT-3′; | | |
| transferase | (SEQ ID NO:17) | | |
| | AS/5′-AGG CTA CGA TGA CGT GCT | | |
| | CAG G-3′; | | |
| | (SEQ ID NO:18) | | |
| Notch 1 | S/5′-GAG TGC CAG CTG ATG CCA | 51 | 293 |
| | AAT G-3′; | | |
| | (SEQ ID NO:19) | | |
| | AS/5′-TTG CCA TTG ACA GGG TTG | | |
| | GTG-3′; | | |
| | (SEQ ID NO:20) | | |
| Sox-1 | S/5′-CCA ACC AGG ACC GGG TCA | 53 | 261 |
| | AA-3′; | | |
| | (SEQ ID NO:21) | | |
| | AS/5′-CCT TCT TGA GCA GCG TCT | | |
| | TGG TC-3′; | | |
| | (SEQ ID NO:22) | | |

Efficacy of both brain and spinal cord NSCs has been compared in SCl model. It has been observed that NSCs from different regions differentiate differently in vivo. Human spinal cord derived cultures were examined to assess whether any particular phenotype (e.g. oligodendrocytes or motor neurons) were enhanced under lower oxygen. Without wishing to be bound by any particular theory, it is believed that NSCs cultured under low oxygen levels are capable of repairing damaged spinal cord in a rat SCl model.

Gene Expression Profile

Gene expression profile, SNP genotyping, HLA typing, Karyotyping and DNA fingerprinting are used to characterize the effects of culturing the cells under low oxygen conditions. Expression profile of NSC-specific genes can be generated. The NSC-specific genes are compared with oxygen-regulated expression of certain family of genes like senescence, stress-induced genes and DNA repair to reveal molecular profile of NSCs grown under two different oxygen levels. NSCs are known to display region and culture condition specific gene expression (Wright et al., 2003, J. Neurochem. 86:179-95). Changes in gene expression are often indicative of changes in underlying biochemical processes. Microarrays are used to analyze global patterns of gene expression. Microarrays are powerful tools for measuring global expression patterns on a large scale. Microarrays or ‘chips’ simultaneously determine expression levels for thousands of genes. Data are then analyzed for patterns of expression that change over various treatments (different oxygen) or time points.

Affymetrix or Illumina® System can be used to analyze gene expression and SNP genotyping. Illumina microarray systems offer remarkably high reproducibility due to 30-fold probe redundancy. Two microarray chip systems allows for gene expression analysis and SNP genotyping. Illumina's Human-6 microchip contains 48,095 probes for gene targets, of which 23,920 are from their HumanRef-8 chip; 11,921 are from UniGene Build; 163,954 are from RefSeq Gnomon; and 2,300 are Genome-Annotation RefSeq. The genomic coverage is sufficient to monitor the expression changes in maintenance of the undifferentiated state.

SNP genotyping and multi-sample gene expression is evaluated using the BeadStation 500GX, with the GoldenGate™ assay. SNP genotyping is accomplished by using SNP multiplexing at 384,768 and 1536-plex, image acquisition and extraction software, automated genotype calling and analysis software, detailed operating procedures and support. This assay system does not require PCR, and enables unlimited multiplexing from a single sample preparation.

HLA Typing

Human leukocyte antigens (HLA) are a family of cell proteins found on the surface of virtually every somatic cell type. The expression of these proteins are of interest for transplantation. Performing HLA typing provides the necessary information to alert clinicians regarding the potential of graft rejection. This would be of importance even in culture systems that do not employ any animal-derived growth and maintenance factors. HLA typing is performed using an adapted hybridization of PCR amplified DNA with sequence specific oligonucleotide probe (SSOP) technology from Tepnel Lifecodes. Genomic DNA is isolated from the NSC cultures using the GenElute™ Mammalian Genomic DNA Miniprep Kit (Sigma, St. Louis, Mo.). Assays are performed to determine the HLA-A, -B, -C, -DRB, and -DQB haplotype for each NSC line.

Karyotypiinz

A normal 2N karyotype is one of the most important requirements for a truly pluripotent and untransformed hESC (Longo et al., 1997, Transgenic Res. 6:321-8.). NSCs are karyotyped every 3-4 passages to guard against the outgrowth of abnormal cells. Karyotyping is performed using a standard G-banding technique. Briefly, cells cultured in T75 culture flasks are treated with 0.05 μg/mL Colcemid and Collagenase IV (Invitrogen, Carlsbad, Calif.) for approximately 2 hours, followed by dissociation using 0.05% trypsin (ATCC 30-2101) in Hank's Balanced Salt Solution (Mediatech, Herndon, Va.). Cells collected by centrifugation (180×g, 10 min), are resuspended in hypotonic KCl solution (0.075 M) for 15 min, and fixed in Carnoy's fixative (3:1, glacial acetic acid: methanol). Metaphase spreads are prepared on glass microscope slides and are exposed briefly to trypsin, stained using a 3:1 Gurr:Giemsa stain, and a minimum of 15 cells are analyzed.

In addition to traditional G-banding, NSCs can be analyzed using SKY spectral karyotyping (Schrock et al., 1997, Hum Genet. 101:255-62). This technique allows for simultaneous visualization of all human chromosomes in 24 different colors (i.e. the whole genome will be analyzed in one hybridization). Also, this technique provides the accurate detection of inter-chromosomal aberrations that may not be detectable by conventional G-banding. Using spectral karyotyping, cryptic translocations and marker chromosomes that may not be definable or characterized by G-banding can be analyzed. In order to use this technology, normal metaphase spread slide preparation is employed as in G-banding. Following this, a standard fluorescent hybridization step using a SkyPaint commercially available ASI kit (Applied Spectral Imaging, Carlsbad, Calif.) is added. The combined use of G-banding and SKY offers a more complete cytogenetic diagnostic capability.

Another cytogenetic technique is fluorescent in situ hybridization (FISH). This technique can be used to confirm suspected chromosomal aberrations, and in order to visualize chromosomes at a higher resolution than permitted by other methods. FISH can be performed using whole chromosome paints, or probes specific for defined chromosome loci. Single gene probes can also be used for FISH.

In order to further characterize the cells cytogenetically, Comparative Genomic Hybridization (CGH) can be performed. CGH is a molecular method used to detect chromosomal aberrations and allows for the comprehensive analysis of multiple DNA gains and losses in entire genomes within a single experiment. CGH detects inversions, deletions, and duplications not detectable by G-banding or SKY spectral karyotyping. In this technique the sample genome is compared to a standard containing control DNA representing each of the chromosomes. Genomic DNA from the sample cell line and normal reference DNA is differentially labeled and simultaneously hybridized in situ to normal metaphase chromosomes. An advantage of molecular karyotyping over traditional methods is that the DNA is analyzed directly by hybridization. No amplification of sample DNA is required prior to hybridization with the array spots. Further, unlike conventional metaphase spread techniques for karyotyping, no harvesting is required before analysis of chromosomal abnormalities. CGH does not detect translocations, which are detectable by G-banding and SKY. By combining all these cytogenetic techniques, many known genomic anomalies can be detected.

DNA Fingerprinting

The discovery of DNA hypervariable regions within genomes has made it possible to validate cell line identity by molecular analysis. It has been demonstrated that hypervariable DNA probes, which consist of tandem repeating units, are capable of hybridizing to many loci distributed throughout the genome to produce a DNA “fingerprint.” Another method is to analyze highly repetitive microsatellite sequences, which are composed of 1 to 6 base pair repeats. Many stable microsatellites in the human genome exhibit multiple alleles which vary in the number of 4 base pair repeats. DNA profiles can be established for each human cell line by characterizing alleles of eight short tandem repeat (STR) loci using ATCC's variation of the Promega PowerPlex 1.2 system. If the two alleles at a given chromosomal locus are the same, the locus is considered homozygous, and heterozygous if the alleles are different. In a homozyogous cell line, the single resulting peak is about double the size of heterozygous peaks since the produced signal is twice as intense. In some instances, a cell line may express three alleles, the result of either gene duplication with subsequent translocation or the emergence of a somatic mutation. Usually, such anomalies are restricted to one locus. If two loci express three or more alleles, then the purity of the cell line is questioned. When three or more loci have greater than heterozygous expression, without a plausible explanation, the sample is considered cross-contaminated with another cell line. The PowerPlex 1.2 System can detect, reproducibly, a cross-contaminating cell line at a level as low as 10%.

This highly sensitive technique requires less than 1.0 ng of test DNA template. The STR markers used for human cell lines are highly specific (i.e. they only amplify human and non-human primate DNA). STR analysis may be performed in 1 to 2 days and the labor required is greatly reduced, compared to conventional methods, due to the fact that the procedure is amenable to automation. The amplified products of STR alleles are usually 100-350 bp. In addition, multiple loci may be analyzed simultaneously, via a multiplex reaction wherein multiple STR loci are amplified simultaneously. The amplitude of each signal is also characteristic of each cell line, and can serve as an indicator of aneuploidy. The level of discrimination for the eight STR markers typically used is approximately 1 in 10⁸. This resolving power ensures that it is highly unlikely that any two established cell lines will have the same profile. The presence of more than two alleles at multiple loci would indicate the possibility of cross contamination, or karyotypic instability among the population of NSC within the tested line.

STR analysis on every sample is performed at least twice. The first analysis occurs as soon as possible in order to verify the identity of the culture. The second analysis is performed on the later passage, again to verify identity and purity, in order to monitor genetic alterations at tested loci. Additional STR analyses for each NSC line can be performed to provide more evidence for genotypic purity and stability of the cell lines and subclones over time.

Routine STR and cytogenetic analyses provides a standard set of assays with which to monitor genetic changes over continued passage in different culturing conditions and validate methods conducive to the maintenance of genomic stability, which is critical for the advancement of stem cell research. In order to confidently and accurately monitor the genotypic purity of the NSC lines, a combination of available technologies are used. For every cell line, STR analysis, G-band karyotyping, SKY spectral karyotyping, and CGH is applied. FISH and STR monoplex can also be used as needed to investigate anomalies at defined chromosomal loci.

Genomic Stability

All dividing cells, including NSCs, undergo spontaneous mutations at a rate of 1 in 10⁹ nucleotides. Since the mutant progeny are likely to represent only a few daughter cells within the culture, they are not detectable as clonal aberrations. An exception to this occurs when the mutation confers a selective growth advantage to the daughter cells, such that the aberrant genotype emerges as the dominant clone. Such clonally detectable genomic aberrancies may also affect the phenotype of the cells involved, particularly if such cells are destined for therapeutic in vivo applications. Genomic analysis for copy number aberrations are performed on an early (P5), intermediate (P10) and late (P15) passage sample from each line.

Affymetrix oligonucleotide arrays containing 115,571 SNPs can be hybridized with genomic DNA from each NSC sample at any given passage. Briefly, 250 ng of genomic DNA are digested with either XbaI or HindIII, adapters are ligated on to the digested DNA, and a generic PCR is performed preferentially amplifying fragments 250-2000 bp in length. Samples are then fragmented, fluorescently labeled, and hybridized to the arrays according to the manufacturer's protocol. Genotypes are determined using the software tool GDAS3.0, with a 0.05 setting for both homozygous and heterozygous genotype calls. Copy number analysis is performed using the Affymetrix GeneChip Chromosome Copy Number Analysis Tool Version2.0 (Huang et al., 2004, Hum Genomics 1:287-99). To reduce noise and potential false positives, SNPs are analyzed in a 10-SNP moving window, each oligonucleotide array is plotted separately, and only changes observed on both arrays are considered to be true positives.

### Example 4

### Manufacture and Banking Human NSCs using Optimal Growth Condition

NSCs generated by the culturing methods discuss herein can be applied to bank NSCs in large quantities. The manufacturing process is validated to ensure consistent results from different batches and different samples.

NSCs are cultured in T75 cm² flasks and 2-stack and 10-stack factories in a Sanyo Multi-gas incubator. NSCs are seeded at 1.5×10⁶ cells/cm² in complete growth medium for 14 days. 50% of the medium is replaced with fresh medium every other day. On day 14, the cells are harvested and the total number of cells/cm² recovered from both vessels are compared. The viability of cells is counted by trypan blue exclusion assay.

In the event that there is an observed difference in expansion rate of NSCs in flask and factories, the gas exchange would need to be validated. It has been observed that when BMSCs were grown in factories, it was observed that there was a difference in gas exchange between flask and factories and as a result when BMSCs were manufactured in factories, 10% CO₂ was needed instead of 5% CO₂. Without wishing to be bound by any particular theory, it is preferred that NSCs are cultured in (i) about 5% CO₂; about 3% O₂, (ii) about 10% CO₂; about 3% O₂, (iii) about 5% CO₂; about 6% O₂, (iv) about 10% CO₂; about 6% O₂. However, it should be appreciated that the cells can be cultured in any combination of CO₂ and O₂ levels, where the level of CO₂ can range from about 5% through about 10% CO₂ and the level of O₂ can range from about 3% through about 6% O₂.

Factory grown NSCs are tested for expression of cell surface markers (including immunological markers) by flow cytometry, differentiation potential into multiple brain lineages by immunocytochemistry and qPCR on differentiated cells. Karyotyping and expression profile can be assessed on the banked cells.

### Example 5

### Growing Human Neural Stem and Progenitor Cells (NSPCs) under Reduced Oxygen

A component of cell based therapy for neurological diseases or injuries such as stroke, spinal cord injury, and traumatic brain injury is the development of a process to manufacture human brain and spinal cord derived neural stem & progenitor cells (NSPCs) in large quantities while maintaining their stem cell state and multipotential to differentiate into neurons, astrocytes and oligodendrocytes. Since NSPCs grow slowly and have limited capacity of expansion, mitogens/growth factors and genetic immortalization have been used to produce large quantities of cells. However, these methods may result in genetic modifications to the cells. Therefore, the following experiments were designed to develop a technique to grow NSPCs in large quantities under physiological conditions without resulting in any substantial genetic modifications of the cells.

Adapting the methods disclosed elsewhere herein, human fetal brain and spinal cord derived neural stem and progenitor cell (NSPC) cultures were established, expanded and characterized. Some of these cells were maintained in culture for more than 20 passages under ambient oxygen (20-21% O₂) and were demonstrated to be multipotent as shown by their ability to differentiate into astrocytes, neurons and oligodendrocytes in vitro under defined differentiation protocols.

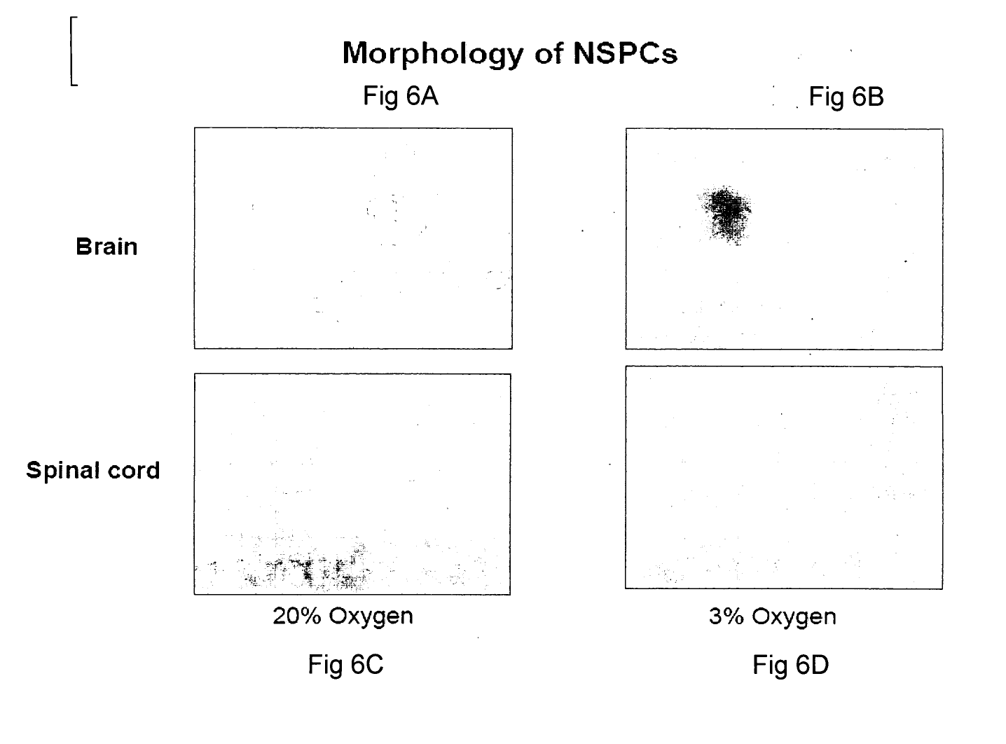

The following experiments were designed to develop more optimum growth conditions for NSPCs. The cells were cultured under several different conditions including reduced oxygen (e.g., 3-6% O₂). It was observed that both brain and spinal cord derived NSPCs cultured under low oxygen conditions (e.g., 3-6% O₂) exhibited a positive effect on cell proliferation rates. These cells grew at least two-three fold faster than cells grown under ambient oxygen (e.g., 20-21% O₂) levels and the highest proliferation was observed when cells were grown on coated dishes under reduced oxygen conditions. In any event, the morphology of neural stem and progenitor cultures was not affected by low oxygen conditions (phase microscopy, see FIG. 6).

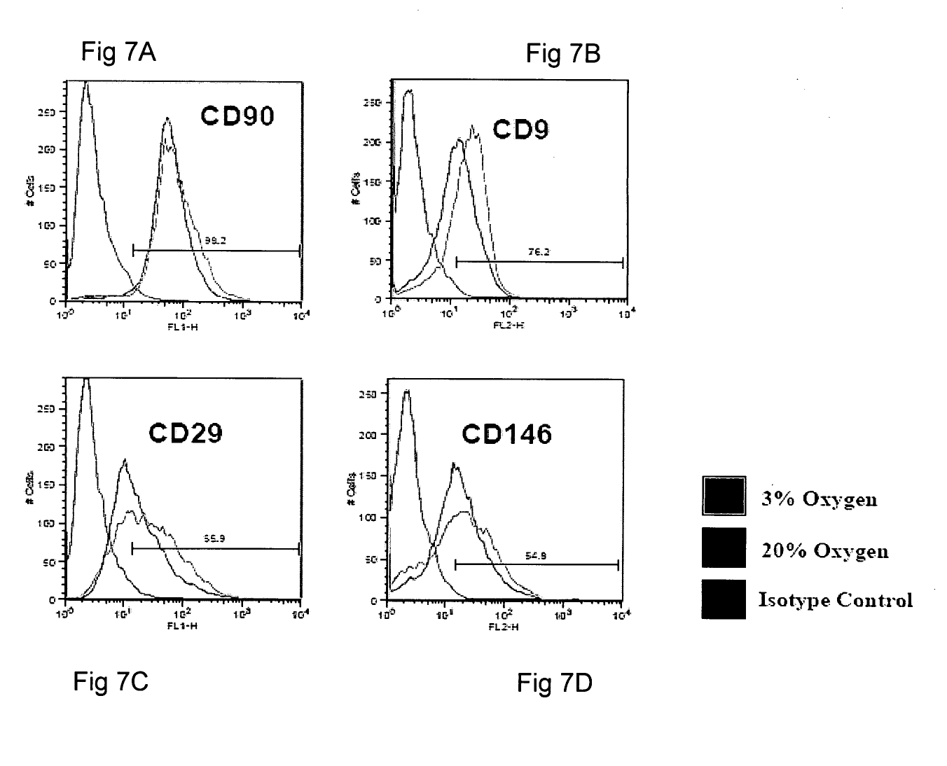

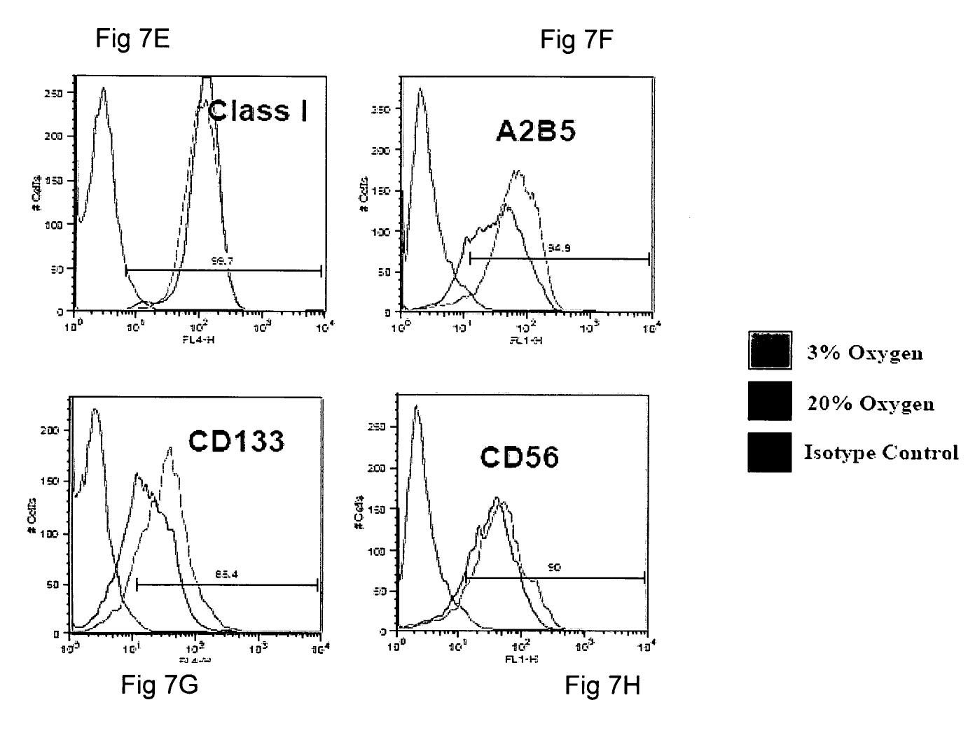

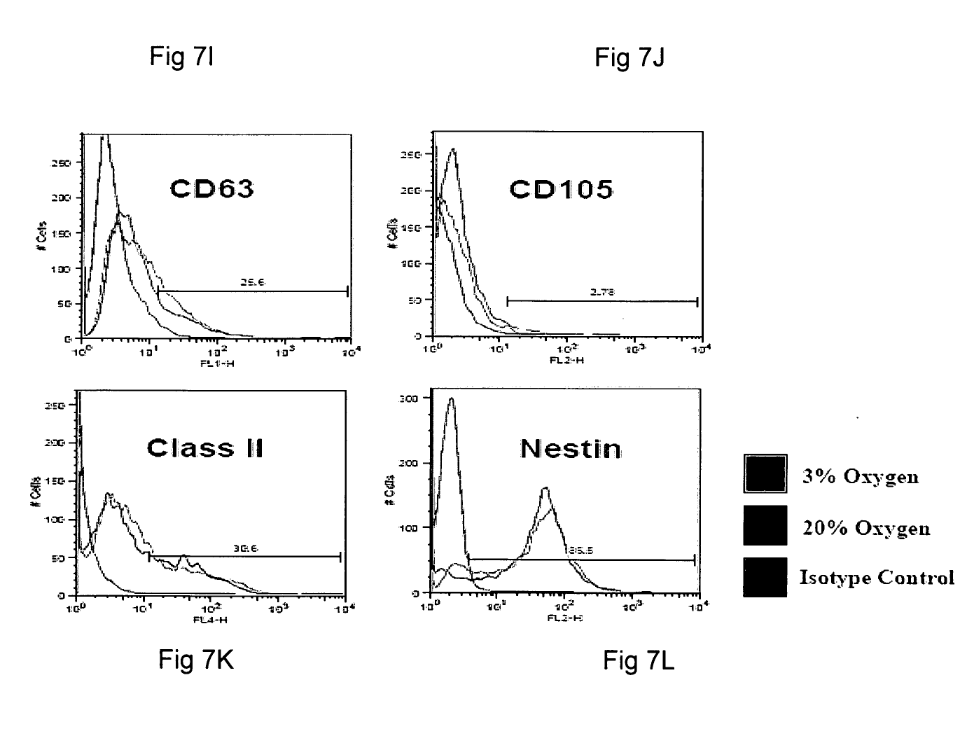

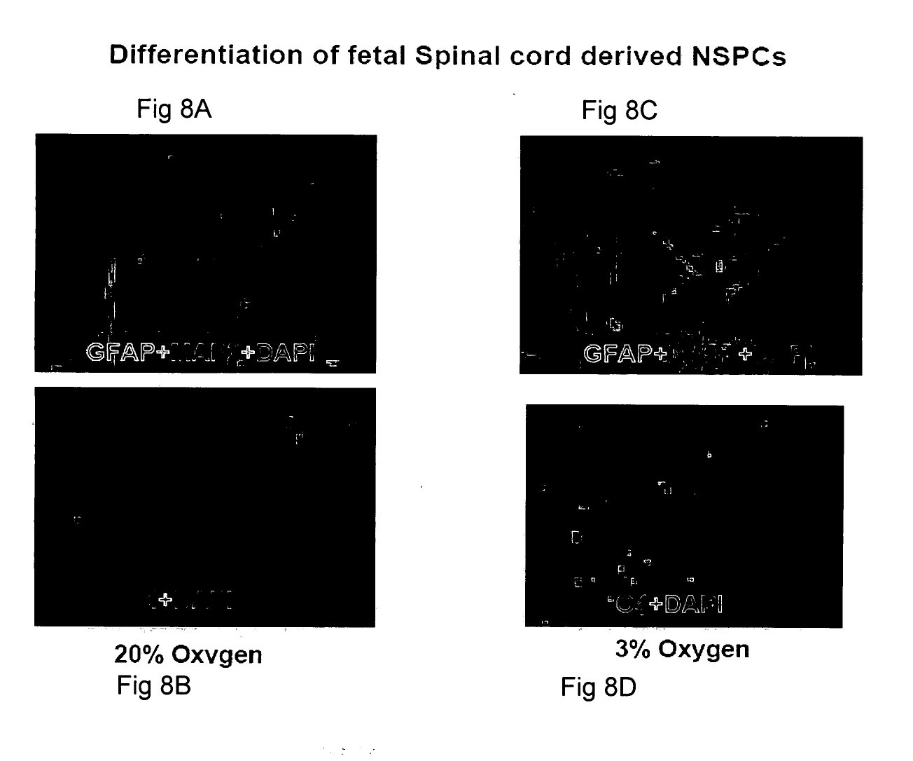

Phenotypic characterization by flow cytometry revealed that there was no significant difference in the expression of nestin, CD133, A2B5, CD56, MHC class I and MHC class II between NSPCs cultured under 3-6% oxygen and 20% oxygen (see FIG. 7). In addition, NSPCs cultured under lower oxygen conditions maintained their multipotentiality and differentiated into oligodendrocytes, neurons and astrocytes under previously defined differentiation protocol in vitro.

With respect to the spinal cord derived NSPC cultures, it was observed that expression of some stem cell specific proteins (CD133) and oligodendrocytes precursors specific proteins (A2B5) as well as CD9 was higher in cells cultured under low oxygen conditions (e.g. 3% oxygen) as measured by flow cytometry (see FIG. 7)

With respect to the brain derived NSPC cultures, expression of SOX8, PCDHB2, WNT1, POU6F1, ROBO1 and CD9 was upregulated, while expression of PDGFC, ACCN1, ERBB2 and DVL3 was down regulated under 3% oxygen (as measured using SuperArray), see Table 3 and Table 4. SOX8 is one of the SRY transcription factor expressed during oligodendrocytes development while WNT1 is involved in number of functions during development including cell fate determination, proliferation and apoptosis. In developing mid-hind brain region, WNT1 controls proliferation specific progenitors.

TABLE 3
Genes Up-regulated (>2-fold) in NSPC
Grown in 3% Oxygen vs. 20% Oxygen

| | | | | | Fold |
|---|---|---|---|---|---|
| | | | | | change |
| | Ref. | | | 20% | (3% |
| Gene Name | Seq. No. | Symbol | 3% O₂ | O₂ | O₂/20% O₂ |
| SRY (sex | NM_014587 | SOX8 | 0.131 | 0.008 | 15.649 |
| determining | | | | | |
| region Y)- | | | | | |
| box 8 | | | | | |
| Protocadherin | NM_018936 | PCDHB2 | 0.148 | 0.014 | 10.674 |
| beta 2 | | | | | |
| Wingless- | NM_005430 | WNT1 | 0.127 | 0.031 | 4.074 |
| type MMTV | | | | | |
| integration | | | | | |
| site family, | | | | | |
| member 1 | | | | | |
| POU domain, | NM_002702 | POU6F1 | 0.091 | 0.022 | 4.063 |
| class 6, | | | | | |
| transcription | | | | | |
| factor 1 | | | | | |
| Roundabout, | NM_002941 | ROBO1 | 0.211 | 0.070 | 3.008 |
| axon | | | | | |
| guidance | | | | | |
| receptor | | | | | |
| homolog 1 | | | | | |
| CD9 antigen | NM_001769 | CD9 | 0.653 | 0.268 | 2.437 |
| (p24) | | | | | |
| S100 calcium | NM_014624 | S100A6 | 0.367 | 0.173 | 2.118 |
| binding | | | | | |
| protein A6 | | | | | |
| (calcyclin) | | | | | |

TABLE 4
Genes Down-regulated (>2-fold) in NSPC Grown in 3% Oxygen vs. 20% Oxygen

| | Ref. Seq. | | | | Fold change |
|---|---|---|---|---|---|
| Gene Name | No. | Symbol | 3% O₂ | 20% O₂ | (3% O₂/20% O₂ |
| Platelet derived | NM_016205 | PDGFC | 0.047 | 0.262 | 0.179 |
| growth factor C | | | | | |
| Amiloride- | NM_001094 | ACCN1 | 0.040 | 0.153 | 0.258 |
| sensitive cation | | | | | |
| channel 1, | | | | | |
| neuronal (degenerin) | | | | | |
| Amyloid beta (A4) | NM_173075 | APBB2 | 0.042 | 0.153 | 0.276 |
| precursor protein- | | | | | |
| binding, family B, | | | | | |
| member 2 (Fe65- | | | | | |
| like) | | | | | |
| CDK5 regulatory | NM_018249 | CDK5RAP2 | 0.055 | 0.168 | 0.331 |
| subunit associated | | | | | |
| protein 2 | | | | | |
| Like- | NM_004737 | LARGE | 0.046 | 0.119 | 0.384 |
| glycosyltransferase | | | | | |
| Forkhead box G1B | NM_005249 | FOXG1B | 0.162 | 0.389 | 0.416 |
| Human 18S | X03205 | 18SrRNA | 0.084 | 0.201 | 0.419 |
| ribosomal RNA | | | | | |
| V-erb-b2 | NM_004448 | ERBB2 | 0.031 | 0.064 | 0.478 |
| erythroblastic | | | | | |
| leukemia viral | | | | | |
| oncogene homolog | | | | | |
| 2, | | | | | |
| neuro/glioblastoma | | | | | |
| derived oncogene | | | | | |
| homolog (avian) | | | | | |
| Dishevelled, dsh | NM_004423 | DVL3 | 0.069 | 0.139 | 0.497 |
| homolog 3 | | | | | |
| (Drosophila) | | | | | |

It was observed that the fate of both brain and spinal cord derived NSPCs was altered under 3% oxygen. In vitro differentiation of both cultures gave rise to more oligodendrocytes (approximately 2-fold).

The disclosures of each and every patent, patent application, and publication cited herein are hereby incorporated herein by reference in their entirety.

It will be apparent to those skilled in the art that various modifications and variations can be made in the methods and compositions of the present invention without departing from the spirit or scope of the invention. Thus, it is intended that the present invention cover the modifications and variations of the present invention provided they come within the scope of the appended claims and their equivalents.

What is claimed:

1. A method of increasing cellular differentiation of an isolated neural stem cell (NSC), the method comprising differentiating said NSC in low oxygen conditions ranging from about 2.5% through about 5% oxygen, wherein said low oxygen conditions increase the cellular differentiation of said NSC when compared with an otherwise identical NSC that is differentiated in ambient oxygen conditions of about 20% oxygen.

2. The method of claim 1, wherein said NSC is derived from a central nervous system tissue selected from the group consisting of brain, spinal cord, or any combinations thereof.

3. The method of claim 1, wherein said cellular differentiation is oligodendrocyte differentiation.

4. The method of claim 1, wherein said NSC is differentiated as an adherent cell on a coated surface.

5. The method of claim 1, wherein said NSC adheres to a surface coated with polyornithine and fibronectin.

6. The method of claim 1, wherein said NSC is a human NSC.

7. The method of claim 1, wherein exogenous genetic material has been introduced into said NSC.

8. The method of claim 1, wherein said cellular differentiation is in the presence of brain-derived neurotrophic factor (BDNF).

9. The method of claim 1, wherein said cellular differentiation is in the presence of insulin-like growth factor 1 (IGF-1).

10. An isolated differentiated neural stem cell (NSC) prepared by a method of differentiating said NSC in low oxygen conditions ranging from about 2.5% through about 5% oxygen, wherein said low oxygen conditions increase the cellular differentiation of said NSC when compared with an otherwise identical NSC that is differentiated under ambient oxygen conditions of about 20% oxygen.

11. The method of claim 10, wherein said NSC is derived from a central nervous system tissue selected from the group consisting of brain, spinal cord, or any combinations thereof.

12. The differentiated NSC of claim 10, wherein said cell expresses a characteristic of an oligodendrocyte, further wherein said characteristic of an oligodendrocyte is the positive staining for O4.

13. The differentiated NSC of claim 10, wherein said NSC is a human NSC.

14. The differentiated NSC of claim 10, wherein exogenous genetic material has been introduced into said NSC.

15. The differentiated NSC of claim 10, wherein said cellular differentiation is in the presence of BDNF.

16. The differentiated NSC of claim 10, wherein said cellular differentiation is in the presence of IGF-1.

17. A method of treating a mammal having a disease, disorder or condition of the central nervous system, the method comprising obtaining an isolated neural stem cell (NSC) from a donor, differentiating said NSC in low oxygen conditions ranging from about 2.5% through about 5% oxygen, and administering said differentiated NSC to the central nervous system of said mammal.

18. The method of claim 17, wherein said NSC is derived from a central nervous system tissue selected from the group consisting of brain, spinal cord, or any combinations thereof.

19. The method of claim 17, wherein said differentiation is in the presence of BDNF.

20. The method of claim 17, wherein said differentiation is in the presence of IGF-1.

21. The method of claim 17, wherein said mammal is a human.

22. The method of claim 17, wherein said isolated NSC is allogeneic with respect to said mammal.

23. The method of claim 17, wherein said isolated NSC is autologous with respect to said mammal.

24. The method of claim 18, wherein said disease, disorder or condition of the central nervous system is selected from the group consisting of a genetic disease, brain trauma, Huntington's disease, Alzheimer's disease, Parkinson's disease, spinal cord injury, stroke, multiple sclerosis, cancer, CNS lysosomal storage diseases, head trauma, and epilepsy.

25. The method of claim 17, wherein said disease, disorder or condition is injury to the tissue or cells of said central nervous system.

26. The method of claim 17, wherein said differentiated NSC administered to said central nervous system remains present in said central nervous system.

27. The method of claim 17, wherein prior to administering said NSC, said NSC is genetically modified.

28. The method of claim 17, wherein said cellular differentiation is oligodendrocyte differentiation.

29. A method of in vitro expansion and maintenance of the multipotentiality of a neural stem cell (NSC), the method comprising culturing said NSC in low oxygen conditions ranging from about 2.5% through about 5% oxygen, wherein said low oxygen conditions increase the cellular proliferation of said NSC when compared with an otherwise identical NSC that is cultured under ambient oxygen conditions of about 20% oxygen.

30. The method of claim 29, wherein said NSC is derived from a central nervous system tissue selected from the group consisting of brain, spinal cord, or any combinations thereof.

31. The method of claim 29, wherein said NSC is cultured as an adherent cell on a coated surface.

32. The method of claim 31, wherein said NSC adheres to a surface coated with polyornithine and fibronectin.

33. The method of claim 30, wherein said NSC is a human NSC.

34. The method of claim 29, wherein exogenous genetic material has been introduced into said NSC.

35. An isolated neural stem cell (NSC) prepared by a method of culturing said NSC in low oxygen conditions ranging from about 2.5% through about 5% oxygen, wherein said low oxygen conditions increase the cellular proliferation of said NSC when compared with an otherwise identical NSC that is cultured under ambient oxygen conditions of about 20% oxygen.

36. The method of claim 35, wherein said NSC is derived from a central nervous system tissue selected from the group consisting of brain, spinal cord, or any combinations thereof.

37. The isolated NSC of claim 35, wherein said NSC is a human NSC.

38. The isolated NSC of claim 35, wherein exogenous genetic material has been introduced into said NSC.

39. A method of treating a mammal having a disease, disorder or condition of the central nervous system, the method comprising obtaining an isolated neural stem cell (NSC) from a donor, culturing said NSC in low oxygen conditions ranging from about 2.5% through about 5% oxygen, and administering said cultured NSC to the central nervous system of said mammal.

40. The method of claim 39, wherein said NSC is derived from a central nervous system tissue selected from the group consisting of brain, spinal cord, or any combinations thereof.

41. The method of claim 39, wherein said mammal is a human.

42. The method of claim 39, wherein said isolated NSC is allogeneic with respect to said mammal.

43. The method of claim 39, wherein said isolated NSC is autologous with respect to said mammal.

44. The method of claim 39, wherein said disease, disorder or condition of the central nervous system is selected from the group consisting of a genetic disease, brain trauma, Huntington's disease, Alzheimer's disease, Parkinson's disease, spinal cord injury, stroke, multiple sclerosis, cancer, CNS lysosomal storage diseases and head trauma, epilepsy.

45. The method of claim 39, wherein said disease, disorder or condition is injury to the tissue or cells of said central nervous system.

46. The method of claim 39, wherein said cultured NSC administered to said central nervous system remains present and/or replicates in said central nervous system.
